SEQUENCE LISTING (28 sequences)

SEQ ID NO: 1 - protein, 843 aa, Homo sapiens
MKVISLFILVGFIGEFQSFSSASSPVNCQWDFYAPWSECNGCTKTQTRRRSVAVYGQYGGQPCVGNAFETQSCEPTRGCPTEEGCGERFRCFSGQCISKSLVCNGDSDCDEDSADEDRCEDSERRPSCDIDKPPPNIELTGNGYNELTGQFRNRVINTKSFGGQCRKVFSGDGKDFYRLSGNVLSYTFQVKINNDFNYEFYNSTWSYVKHTSTEHTSSSRKRSFFRSSSSSSRSYTSHTNEIHKGKSYQLLVVENTVEVAQFINNNPEFLQLAEPFWKELSHLPSLYDYSAYRRLIDQYGTHYLQSGSLGGEYRVLFYVDSEKLKQNDFNSVEEKKCKSSGWHFVVKFSSHGCKELENALKAASGTQNNVLRGEPFIRGGGAGFISGLSYLELDNPAGNKRRYSAWAESVTNLPQVIKQKLTPLYELVKEVPCASVKKLYLKWALEEYLDEFDPCHCRPCQNGGLATVEGTHCLCHCKPYTFGAACEQGVLVGNQAGGVDGGWSCWSSWSPCVQGKKTRSRECNNPPPSGGGRSCVGETTESTQCEDEELEHLRLLEPHCFPLSLVPTEFCPSPPALKDGFVQDEGTMFPVGKNVVYTCNEGYSLIGNPVARCGEDLRWLVGEMHCQKIACVLPVLMDGIQSHPQKPFYTVGEKVTVSCSGGMSLEGPSAFLCGSSLKWSPEMKNARCVQKENPLTQAVPKCQRWEKLQNSRCVCKMPYECGPSLDVCAQDERSKRILPLTVCKMHVLHCQGRNYTLTGRDSCTLPASAEKACGACPLWGKCDAESSKCVCREASECEEEGFSICVEVNGKEQTMSECEAGALRCRGQSISVTSIRPCAAETQ

SEQ ID NO: 2 - DNA, 4015 bp, Homo sapiens
ggcagcctgctgggctcttcctgctgttgaaaacttacccggcccttacagaggaaatcttcctcctctcttctgccctgaatgttttcccaaacatgaaggtgataagcttattcattttggtgggatttataggagagttccaaagtttttcaagtgcctcctctccagtcaactgccagtgggacttctatgccccttggtcagaatgcaatggctgtaccaagactcagactcgcaggcggtcagttgctgtgtatgggcagtatggaggccagccttgtgttggaaatgcttttgaaacacagtcctgtgaacctacaagaggatgtccaacagaggagggatgtggagagcgtttcaggtgcttttcaggtcagtgcatcagcaaatcattggtttgcaatggggattctgactgtgatgaagacagtgctgatgaagacagatgtgaggactcagaaaggagaccttcctgtgatatcgataaacctcctcctaacatagaacttactggaaatggttacaatgaactcactggccagtttaggaacagagtcatcaataccaaaagttttggtggtcaatgtagaaaggtgtttagtggggatggaaaagatttctacaggctgagtggaaatgtcctgtcctatacattccaggtgaaaataaataatgattttaattatgaattttacaatagtacttggtcttatgtaaaacatacgtcgacagaacacacatcatctagtcggaagcgctccttttttagatcttcatcatcttcttcacgcagttatacttcacataccaatgaaatccataaaggaaagagttaccaactgctggttgttgagaacactgttgaagtggctcagttcattaataacaatccagaatttttacaacttgctgagccattctggaaggagctttcccacctcccctctctgtatgactacagtgcctaccgaagattaatcgaccagtacgggacacattatctgcaatctgggtcgttaggaggagaatacagagttctattttatgtggactcagaaaaattaaaacaaaatgattttaattcagtcgaagaaaagaaatgtaaatcctcaggttggcattttgtcgttaaattttcaagtcatggatgcaaggaactggaaaacgctttaaaagctgcttcaggaacccagaacaatgtattgcgaggagaaccgttcatcagagggggaggtgcaggcttcatatctggccttagttacctagagctggacaatcctgctggaaacaaaaggcgatattctgcctgggcagaatctgtgactaatcttcctcaagtcataaaacaaaagctgacacctttatatgagctggtaaaggaagtaccttgtgcctctgtgaaaaaactatacctgaaatgggctcttgaagagtatctggatgaatttgacccctgtcattgccggccttgtcaaaatggtggtttggctactgttgaggggacccattgtctgtgccattgcaaaccgtacacatttggtgcggcgtgtgagcaaggagtcctcgtagggaatcaagcaggaggggttgatggaggttggagttgctggtcctcttggagcccctgtgtccaagggaagaaaacaagaagccgtgaatgcaataacccacctcccagtgggggtgggagatcctgcgttggagaaacgacagaaagcacacaatgcgaagatgaggagctggagcacttgaggttgcttgaaccacattgctttcctttgtctttggttccaacagaattctgtccatcacctcctgccttgaaagatggatttgttcaagatgaaggtacaatgtttcctgtggggaaaaatgtagtgtacacttgcaatgaaggatactctcttattggaaacccagtggccagatgtggagaagatttacggtggcttgttggggaaatgcattgtcagaaaattgcctgtgttctacctgtactgatggatggcatacagagtcacccccaaaaacctttctacacagttggtgagaaggtgactgtttcctgttcaggtggcatgtccttagaaggtccttcagcatttctctgtggctccagccttaagtggagtcctgagatgaagaatgcccgctgtgtacaaaaagaaaatccgttaacacaggcagtgcctaaatgtcagcgctgggagaaactgcagaattcaagatgtgtttgtaaaatgccctacgaatgtggaccttccttggatgtatgtgctcaagatgagagaagcaaaaggatactgcctctgacagtttgcaagatgcatgttctccactgtcagggtagaaattacacccttactggtagggacagctgtactctgcctgcctcagctgagaaagcttgtggtgcctgcccactgtggggaaaatgtgatgctgagagcagcaaatgtgtctgccgagaagcatcggagtgcgaggaagaagggtttagcatttgtgtggaagtgaacggcaaggagcagacgatgtctgagtgtgaggcgggcgctctgagatgcagagggcagagcatctctgtcaccagcataaggccttgtgctgcggaaacccagtaggctcctggaggccctggtcagcttgcttggaatccagcaggcagctggggctgagtgaaaacatctgcacaactgggcactggacagcttttccttcttctccagtgtctaccttcctcctcaactcccagccatctgtataaacacaatcctttgttctcccaaatctgaatcgaattactcttttgcctcctttttaatgtcagtaaggatatgagcctttgcacaggctggctgcgtgttcttgaaataggtgttaccttctctgggccttggttttttaaaatctgtaaaattagaggattgcactagagaaacttgaatgctccattcaggcctatcattttattaagtatgattgacacagcccatgggccagaacacactctacaaaatgactaggataacagaaagaacgtgatctcctgattagagagggtggttttcctcaatggaaccaaatataaagaggacttgaacaaaaatgacagatacaaactatttctatcctgagtagtaatctcacacttcatcctatagagtcaaccaccacagataggaattccttattctttttttaatttttttaagacagagtctcactttgttgcccaggctggagcgcagtggggtgatctcatctccctgcaacctccgcctcctgggttcaagcgattcttgtgcctcagcttcccaagcagctgggattacaggtgcccgccaccacgcccagctaatttttgcatttttagtagagatggggtttcaccatgttggccacgctcgtctccaactcctgacctcaggtaatccgcctgccttggcctcccaaagtgctgggattacagacatgaaccaccacgcctggctggaatacttactcttgtcgggagattgaaccactaaaatgttagagcagaattcattatgctgtggtcacaggggtgtcttgtctgagaacaaatacaattcagtcttctctttggggttttagtatgtgtcaaacataggactggaagtttgcccctgttcttttttcttttgaaagaacatcagttcatgcctgaggcatgagtgactgtgcatttgagaatagttttccctattctgtggatacagtcccagagttttcagggagtacacaggtagattagtttgaagcattgaccttttatttattccttatttctctttcatcaaaacaaaacagcagctgtgggaggagaaatgagagggcttaaatgaaatttaaaataagctatattatacaaatactatctctgtattgttctgaccctggtaaatatatttcaaaacttcagatgacaaggattagaacactcattaaagatgctattcttcagaaaaaaaaaaaaaaaaaaaaaaaa

SEQ ID NO: 3 - protein, 98 aa, Homo sapiens
MIPGGLSEAKPATPEIQEIVDKVKPQLEEKTNETYGKLEAVQYKTQVVAGTNYYIKVRAGDNKYMHLKVFKSLPGQNEDLVLTGYQVDKNKDDELTGF

SEQ ID NO: 4 - DNA, 838 bp, Homo sapiens
tgctgtttgtggaaaataaagcattctataggcggagctagtgaacgcctcttttaaaacacgagtctccacacttccctgttcactttggttccagcatcctgtccagcaaagaagcaatcagccaaaatgatacctggaggcttatctgaggccaaacccgccactccagaaatccaggagattgttgataaggttaaaccacagcttgaagaaaaaacaaatgagacttacggaaaattggaagctgtgcagtataaaactcaagttgttgctggaacaaattactacattaaggtacgagcaggtgataataaatatatgcacttgaaagtattcaaaagtcttcccggacaaaatgaggacttggtacttactggataccaggttgacaaaaacaaggatgacgagctgacgggcttttagcagcatgtacccaaagtgttctgattccttcaactggctactgagtcatgatccttgctgataaatataaccatcaataaagaagcattcttttccaaagaaattatttcttcaattatttctcatttattgtattaagcagaaattaccttttctttctcaaaatcagtgttattgctttagagtataaactccatataaattgatggcaattggaaatcttataaaaactagtcaagcctaatgcaactggctaaaggatagtaccaccctcacccccaccataggcaggctggatcgtggactatcaattcaccagcctccttgttccctgtggctgctgataacccaacattccatctctaccctcatacttcaaaattaaatcaagtattttacaaaaaaaaaaaaa

SEQ ID NO: 5 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 6 - DNA, 3340 bp, Homo sapiens
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaaggtgggacattgctgctgctgctcactcagttccacagcagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 7 - protein, 597 aa, Homo sapiens
MTPIVTVLICLGLSLGPRTRVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVMAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 8 - DNA, 2799 bp, Homo sapiens
ggggaagccactgctaccctcatcaggaagggcagacacaagaagcaccagttctatttgctgctacatcccggctctcgcaccgagggctcatccatccgcagagcagggcagtgggaggagacgccatgacccccatcgtcacagtcctgatctgtctcgggctgagtctgggccccaggacccgcgtgcagacagggaccatccccaagcccaccctgtgggctgagccagactctgtgatcacccaggggagtcccgtcaccctcagttgtcaggggagccttgaagcccaggagtaccgtctatatagggagaaaaaatcagcatcttggattacacggatacgaccagagcttgtgaagaacggccagttccacatcccatccatcacctgggaacacacagggcgatatggctgtcagtattacagccgcgctcggtggtctgagctcagtgaccccctggtgctggtgatgacaggagcctacccaaaacccaccctctcagcccagcccagccctgtggtgacctcaggaggaagggtgaccctccagtgtgagtcacaggtggcatttggcggcttcattctgtgtaaggaaggagaagatgaacacccacaatgcctgaactcccagccccatgcccgtgggtcgtcccgcgccatcttctccgtgggccccgtgagcccgaatcgcaggtggtcgcacaggtgctatggttatgacttgaactctccctatgtgtggtcttcacccagtgatctcctggagctcctggtcccaggtgtttctaagaagccatcactctcagtgcagccgggtcctgtcatggcccctggggaaagcctgaccctccagtgtgtctctgatgtcggctatgacagatttgttctgtacaaggagggggaacgtgaccttcgccagctccctggccggcagccccaggctgggctctcccaggccaacttcaccctgggccctgtgagccgctcctacgggggccagtacagatgctacggtgcacacaacctctcctctgagtgctcggcccccagcgaccccctggacatcctgatcacaggacagatccgtggcacacccttcatctcagtgcagccaggccccacagtggcctcaggagagaacgtgaccctgctgtgtcagtcatggcggcagttccacactttccttctgaccaaggcgggagcagctgatgccccactccgtctaagatcaatacacgaatatcctaagtaccaggctgaattccccatgagtcctgtgacctcagcccacgcggggacctacaggtgctacggctcactcaactccgacccctacctgctgtctcaccccagtgagcccctggagctcgtggtctcaggaccctccatgggttccagccccccacccaccggtcccatctccacacctggccctgaggaccagcccctcacccccactgggtcggatccccaaagtggtctgggaaggcacctgggggttgtgatcggcatcttggtggccgtcgtcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgacgcccaggaagaaaacctctatgctgccgtgaaggacacacagcctgaagatggggtggagatggacactcgggctgctgcatctgaagccccccaggatgtgacctacgcccagctgcacagcttgaccctcagacggaaggcaactgagcctcctccatcccaggaaagggaacctccagctgagcccagcatctacgccaccctggccatccactagcccggagggtacgcagactccacactcagtagaaggagactcaggactgctgaaggcacgggagctgcccccagtggacaccaatgaaccccagtcagcctggacccctaacaaagaccatgaggagatgctgggaactttgggactcacttgattctgcagtcgaaataactaatatccctacattttttaattaaagcaacagacttctcaataatcaatgagttaaccgagaaaactaaaatcagaagtaagaatgtgctttaaactgaatcacaatataaatattacacatcacacaatgaaattgaaaaagtacaaaccacaaatgaaaaaagtagaaacgaaaaaaaaaaactaggaaatgaatgacgttggctttcgtataaggaatttagaaaaagaataaccaattattccaaatgaaggtgtaagaaagggaataagaagaagaagagttgctcatgaggaaaaaccaaaacttgaaaattcaacaaagccaatgaagctcattcttgaaaatattaattacagtcataaatcctaactacattgagcaagagaaagaaagagcaggcacgcatttccatatgggagtgagccagcagacagcccagcagatcctacacacattttcacaaactaaccccagaacaggctgcaaacctataccaatatactagaaaatgcagattaaatggatgaaatattcaaaactggagtttacataatgaacgtaagagtaatcagagaatctgactcattttaaatgtgtgtgtatgtgtgtgtatatatatgtgtgtgtgtgtgtgtgtgtgtgtgtgtgaaaaacattgactgtaataaaaatgttcccatcgtaaaaaaaaaaaaaaaa

SEQ ID NO: 9 - protein, 346 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 10 - DNA, 1038 bp, Homo sapiens
atggcaatggtatcagaattcctcaagcaggcctggtttattgaaaatgaagagcaggaatatgttcaaactgtgaagtcatccaaaggtggtcccggatcagcggtgagcccctatcctaccttcaatccatcctcggatgtcgctgccttgcataaggccataatggttaaaggtgtggatgaagcaaccatcattgacattctaactaagcgaaacaatgcacagcgtcaacagatcaaagcagcatatctccaggaaacaggaaagcccctggatgaaacactgaagaaagcccttacaggtcaccttgaggaggttgttttagctctgctaaaaactccagcgcaatttgatgctgatgaacttcgtgctgccatgaagggccttggaactgatgaagatactctaattgagattttggcatcaagaactaacaaagaaatcagagacattaacagggtctacagagaggaactgaagagagatctggccaaagacataacctcagacacatctggagattttcggaacgctttgctttctcttgctaagggtgaccgatctgaggactttggtgtgaatgaagacttggctgattcagatgccagggccttgtatgaagcaggagaaaggagaaaggggacagacgtaaacgtgttcaataccatccttaccaccagaagctatccacaacttcgcagagtgtttcagaaatacaccaagtacagtaagcatgacatgaacaaagttctggacctggagttgaaaggtgacattgagaaatgcctcacagctatcgtgaagtgcgccacaagcaaaccagctttctttgcagagaagcttcatcaagccatgaaaggtgttggaactcgccataaggcattgatcaggattatggtttcccgttctgaaattgacatgaatgatatcaaagcattctatcagaagatgtatggtatctccctttgccaagccatcctggatgaaaccaaaggagattatgagaaaatcctggtggctctttgtggaggaaac

SEQ ID NO: 11 - protein, 339 aa, Homo sapiens
MSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 12 - DNA, 1500 bp, Homo sapiens
gctcagcatttggggacgctctcagctctcggcgcacggcccagggtgaaaatgtttgccattaaactcacatgaagtaggaaatatttatatggatacaaaaggcacctgcatgggataatgtcaaatttcatagatactgctttgtgcttccttcaaaatgtctactgttcacgaaatcctgtgcaagctcagcttggagggtgatcactctacacccccaagtgcatatgggtctgtcaaagcctatactaactttgatgctgagcgggatgctttgaacattgaaacagccatcaagaccaaaggtgtggatgaggtcaccattgtcaacattttgaccaaccgcagcaatgcacagagacaggatattgccttcgcctaccagagaaggaccaaaaaggaacttgcatcagcactgaagtcagccttatctggccacctggagacggtgattttgggcctattgaagacacctgctcagtatgacgcttctgagctaaaagcttccatgaaggggctgggaaccgacgaggactctctcattgagatcatctgctccagaaccaaccaggagctgcaggaaattaacagagtctacaaggaaatgtacaagactgatctggagaaggacattatttcggacacatctggtgacttccgcaagctgatggttgccctggcaaagggtagaagagcagaggatggctctgtcattgattatgaactgattgaccaagatgctcgggatctctatgacgctggagtgaagaggaaaggaactgatgttcccaagtggatcagcatcatgaccgagcggagcgtgccccacctccagaaagtatttgataggtacaagagttacagcccttatgacatgttggaaagcatcaggaaagaggttaaaggagacctggaaaatgctttcctgaacctggttcagtgcattcagaacaagcccctgtattttgctgatcggctgtatgactccatgaagggcaaggggacgcgagataaggtcctgatcagaatcatggtctcccgcagtgaagtggacatgttgaaaattaggtctgaattcaagagaaagtacggcaagtccctgtactattatatccagcaagacactaagggcgactaccagaaagcgctgctgtacctgtgtggtggagatgactgaagcccgacacggcctgagcgtccagaaatggtgctcaccatgcttccagctaacaggtctagaaaaccagcttgcgaataacagtccccgtggccatccctgtgagggtgacgttagcattacccccaacctcattttagttgcctaagcattgcctggccttcctgtctagtctctcctgtaagccaaagaaatgaacattccaaggagttggaagtgaagtctatgatgtgaaacactttgcctcctgtgtactgtgtcataaacagatgaataaactgaatttgtacaaaaaaaaaaaaaaaaaaaaaaaaaaaaaa

SEQ ID NO: 13 - protein, 236 aa, Homo sapiens
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVTRTVNTAVAITLACFGLAREGNHKPIDYLNPPK

SEQ ID NO: 14 - DNA, 1216 bp, Homo sapiens
gtcactgagggttgactgactggagagctcaagtgcagcaaagagaagtgtcagagcatgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtaactcgaacagtgaacacagcagttgcaataaccttggcttgtttcggacttgctcgggagggtaatcacaagcctattgactaccttaacccacctaagtaaatgtggaaacatccgatataaatctcatagttaatggcataattagaaagctaatcattttcttaagcatagagttatccttctaaagacttgttctttcagaaaaatgtttttccaattagtataaactctacaaattccctcttggtgtaaaattcaagatgtggaaattctaacttttttgaaatttaaaagcttatattttctaacttggcaaaagacttatccttagaaagagaagtgtacattgatttccaattaaaaatttgctggcattaaaaataagcacacttacataagcccccatacatagagtgggactcttggaatcaggagacaaagctaccacatgtggaaaggtactatgtgtccatgtcattcaaaaaatgtgattttttataataaactctttataactaaaaaaaaaaaaaaaaaaaaaaaaaaaaaa

SEQ ID NO: 15 - protein, 330 aa, Homo sapiens
MSAKSRTIGIIGAPFSKGQPRGGVEEGPTVLRKAGLLEKLKEQVTQNFLILECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 16 - DNA, 1499 bp, Homo sapiens
ggaaaaaaaagatgcgccctctgtcactgagggttgactgactggagagctcaagtgcagcaaagagaagtgtcagagcatgagcgccaagtccagaaccatagggattattggagctcctttctcaaagggacagccacgaggaggggtggaagaaggccctacagtattgagaaaggctggtctgcttgagaaacttaaagaacaagtaactcaaaactttttaattttagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaaatgtggaaacatccgatataaatctcatagttaatggcataattagaaagctaatcattttcttaagcatagagttatccttctaaagacttgttctttcagaaaaatgtttttccaattagtataaactctacaaattccctcttggtgtaaaattcaagatgtggaaattctaacttttttgaaatttaaaagcttatattttctaacttggcaaaagacttatccttagaaagagaagtgtacattgatttccaattaaaaatttgctggcattaaaaataagcacacttacataagcccccatacatagagtgggactcttggaatcaggagacaaagctaccacatgtggaaaggtactatgtgtccatgtcattcaaaaaatgtgattttttataataaactctttataacaagattaaaa

SEQ ID NO: 17 - protein, 322 aa, Homo sapiens
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 18 - DNA, 1475 bp, Homo sapiens
ggaaaaaaaagatgcgccctctgtcactgagggttgactgactggagagctcaagtgcagcaaagagaagtgtcagagcatgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaaatgtggaaacatccgatataaatctcatagttaatggcataattagaaagctaatcattttcttaagcatagagttatccttctaaagacttgttctttcagaaaaatgtttttccaattagtataaactctacaaattccctcttggtgtaaaattcaagatgtggaaattctaacttttttgaaatttaaaagcttatattttctaacttggcaaaagacttatccttagaaagagaagtgtacattgatttccaattaaaaatttgctggcattaaaaataagcacacttacataagcccccatacatagagtgggactcttggaatcaggagacaaagctaccacatgtggaaaggtactatgtgtccatgtcattcaaaaaatgtgattttttataataaactctttataacaagattaaaa

SEQ ID NO: 19 - protein, 1744 aa, Homo sapiens
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAATAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRGSSTWLTAFVLKVLSLAQEQVGGSPEKLQETSNWLLSQQQADGSFQDLSPVIHRSMQGGLVGNDETVALTAFVTIALHHGLAVFQDEGAEPLKQRVEASISKASSFLGEKASAGLLGAHAAAITAYALTLTKAPADLRGVAHNNLMAMAQETGDNLYWGSVTGSQSNAVSPTPAPRNPSDPMPQAPALWIETTAYALLHLLLHEGKAEMADQAAAWLTRQGSFQGGFRSTQDTVIALDALSAYWIASHTTEERGLNVTLSSTGRNGFKSHALQLNNRQIRGLEEELQFSLGSKINVKVGGNSKGTLKVLRTYNVLDMKNTTCQDLQIEVTVKGHVEYTMEANEDYEDYEYDELPAKDDPDAPLQPVTPLQLFEGRRNRRRREAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 20 - DNA, 5444 bp, Homo sapiens
agaaggtagcagacagacagacggatctaacctctcttggatcctccagccatgaggctgctctgggggctgatctgggcatccagcttcttcaccttatctctgcagaagcccaggttgctcttgttctctccttctgtggttcatctgggggtccccctatcggtgggggtgcagctccaggatgtgccccgaggacaggtagtgaaaggatcagtgttcctgagaaacccatctcgtaataatgtcccctgctccccaaaggtggacttcacccttagctcagaaagagacttcgcactcctcagtctccaggtgcccttgaaagatgcgaagagctgtggcctccatcaactcctcagaggccctgaggtccagctggtggcccattcgccatggctaaaggactctctgtccagaacgacaaacatccagggtatcaacctgctcttctcctctcgccgggggcacctctttttgcagacggaccagcccatttacaaccctggccagcgggttcggtaccgggtctttgctctggatcagaagatgcgcccgagcactgacaccatcacagtcatggtggagaactctcacggcctccgcgtgcggaagaaggaggtgtacatgccctcgtccatcttccaggatgactttgtgatcccagacatctcagagccagggacctggaagatctcagcccgattctcagatggcctggaatccaacagcagcacccagtttgaggtgaagaaatatgtccttcccaactttgaggtgaagatcacccctggaaagccctacatcctgacggtgccaggccatcttgatgaaatgcagttagacatccaggccaggtacatctatgggaagccagtgcagggggtggcatatgtgcgctttgggctcctagatgaggatggtaagaagactttctttcgggggctggagagtcagaccaagctggtgaatggacagagccacatttccctctcaaaggcagagttccaggacgccctggagaagctgaatatgggcattactgacctccaggggctgcgcctctacgttgctgcagccatcattgagtctccaggtggggagatggaggaggcagagctcacatcctggtattttgtgtcatctcccttctccttggatcttagcaagaccaagcgacaccttgtgcctggggcccccttcctgctgcaggccttggtccgtgagatgtcaggctccccagcttctggcattcctgtcaaagtttctgccacggtgtcttctcctgggtctgttcctgaagtccaggacattcagcaaaacacagacgggagcggccaagtcagcattccaataattatccctcagaccatctcagagctgcagctctcagtatctgcaggctccccacatccagcgatagccaggctcactgtggcagccccaccttcaggaggccccgggtttctgtctattgagcggccggattctcgacctcctcgtgttggggacactctgaacctgaacttgcgagccgtgggcagtggggccaccttttctcattactactacatgatcctatcccgagggcagatcgtgttcatgaatcgagagcccaagaggaccctgacctcggtctcggtgtttgtggaccatcacctggcaccctccttctactttgtggccttctactaccatggagaccacccagtggccaactccctgcgagtggatgtccaggctggggcctgcgagggcaagctggagctcagcgtggacggtgccaagcagtaccggaacggggagtccgtgaagctccacttagaaaccgactccctagccctggtggcgctgggagccttggacacagctctgtatgctgcaggcagcaagtcccacaagcccctcaacatgggcaaggtctttgaagctatgaacagctatgacctcggctgtggtcctgggggtggggacagtgcccttcaggtgttccaggcagcgggcctggccttttctgatggagaccagtggaccttatccagaaagagactaagctgtcccaaggagaagacaacccggaaaaagagaaacgtgaacttccaaaaggcgattaatgagaaattgggtcagtatgcttccccgacagccaagcgctgctgccaggatggggtgacacgtctgcccatgatgcgttcctgcgagcagcgggcagcccgcgtgcagcagccggactgccgggagcccttcctgtcctgctgccaatttgctgagagtctgcgcaagaagagcagggacaagggccaggcgggcctccaacgagccctggagatcctgcaggaggaggacctgattgatgaggatgacattcccgtgcgcagcttcttcccagagaactggctctggagagtggaaacagtggaccgctttcaaatattgacactgtggctccccgactctctgaccacgtgggagatccatggcctgagcctgtccaaaaccaaaggcctatgtgtggccaccccagtccagctccgggtgttccgcgagttccacctgcacctccgcctgcccatgtctgtccgccgctttgagcagctggagctgcggcctgtcctctataactacctggataaaaacctgactgtgagcgtccacgtgtccccagtggaggggctgtgcctggctgggggcggagggctggcccagcaggtgctggtgcctgcgggctctgcccggcctgttgccttctctgtggtgcccacggcagccaccgctgtgtctctgaaggtggtggctcgagggtccttcgaattccctgtgggagatgcggtgtccaaggttctgcagattgagaaggaaggggccatccatagagaggagctggtctatgaactcaaccccttggaccaccgaggccggaccttggaaatacctggcaactctgatcccaatatgatccctgatggggactttaacagctacgtcagggttacagcctcagatccattggacactttaggctctgagggggccttgtcaccaggaggcgtggcctccctcttgaggcttcctcgaggctgtggggagcaaaccatgatctacttggctccgacactggctgcttcccgctacctggacaagacagagcagtggagcacactgcctcccgagaccaaggaccacgccgtggatctgatccagaaaggctacatgcggatccagcagtttcggaaggcggatggttcctatgcggcttggttgtcacggggcagcagcacctggctcacagcctttgtgttgaaggtcctgagtttggcccaggagcaggtaggaggctcgcctgagaaactgcaggagacatctaactggcttctgtcccagcagcaggctgacggctcgttccaggacctctctccagtgatacataggagcatgcaggggggtttggtgggcaatgatgagactgtggcactcacagcctttgtgaccatcgcccttcatcatgggctggccgtcttccaggatgagggtgcagagccattgaagcagagagtggaagcctccatctcaaaggcaagctcatttttgggggagaaagcaagtgctgggctcctgggtgcccacgcagctgccatcacggcctatgccctgacactgaccaaggcccctgcggacctgcggggtgttgcccacaacaacctcatggcaatggcccaggagactggagataacctgtactggggctcagtcactggttctcagagcaatgccgtgtcgcccaccccggctcctcgcaacccatccgaccccatgccccaggccccagccctgtggattgaaaccacagcctacgccctgctgcacctcctgcttcacgagggcaaagcagagatggcagaccaggctgcggcctggctcacccgtcagggcagcttccaagggggattccgcagtacccaagacacggtgattgccctggatgccctgtctgcctactggattgcctcccacaccactgaggagaggggtctcaatgtgactctcagctccacaggccggaatgggttcaagtcccacgcgctgcagctgaacaaccgccagattcgcggcctggaggaggagctgcagttttccttgggcagcaagatcaatgtgaaggtgggaggaaacagcaaaggaaccctgaaggtccttcgtacctacaatgtcctggacatgaagaacacgacctgccaggacctacagatagaagtgacagtcaaaggccacgtcgagtacacgatggaagcaaacgaggactatgaggactatgagtacgatgagcttccagccaaggatgacccagatgcccctctgcagcccgtgacacccctgcagctgtttgagggtcggaggaaccgccgcaggagggaggcgcccaaggtggtggaggagcaggagtccagggtgcactacaccgtgtgcatctggcggaacggcaaggtggggctgtctggcatggccatcgcggacgtcaccctcctgagtggattccacgccctgcgtgctgacctggagaagctgacctccctctctgaccgttacgtgagtcactttgagaccgaggggccccacgtcctgctgtattttgactcggtccccacctcccgggagtgcgtgggctttgaggctgtgcaggaagtgccggtggggctggtgcagccggccagcgcaaccctgtacgactactacaaccccgagcgcagatgttctgtgttttacggggcaccaagtaagagcagactcttggccaccttgtgttctgctgaagtctgccagtgtgctgaggggaagtgccctcgccagcgtcgcgccctggagcggggtctgcaggacgaggatggctacaggatgaagtttgcctgctactacccccgtgtggagtacggcttccaggttaaggttctccgagaagacagcagagctgctttccgcctctttgagaccaagatcacccaagtcctgcacttcaccaaggatgtcaaggccgctgctaatcagatgcgcaacttcctggttcgagcctcctgccgccttcgcttggaacctgggaaagaatatttgatcatgggtctggatggggccacctatgacctcgagggacacccccagtacctgctggactcgaatagctggatcgaggagatgccctctgaacgcctgtgccggagcacccgccagcgggcagcctgtgcccagctcaacgacttcctccaggagtatggcactcaggggtgccaggtgtgagggctgccctcccacctccgctgggaggaacctgaacctgggaaccatgaagctggaagcactgctgtgtccgctttcatgaacacagcctgggaccagggcatattaaaggcttttggcagcaaagtgtcagtgttggcaaaaaaaaaaaaaaaaaa

SEQ ID NO: 21 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 22 - DNA, 1365 bp, Homo sapiens
cttccagaggcaagaccaaccaagatgagtgccctgggagctgtcattgccctcctgctctggggacagctttttgcagtggactcaggcaatgatgtcacggatatcgcagatgacggctgcccgaagccccccgagattgcacatggctatgtggagcactcggttcgctaccagtgtaagaactactacaaactgcgcacagaaggagatggagtatacaccttaaatgataagaagcagtggataaataaggctgttggagataaacttcctgaatgtgaagcagatgacggctgcccgaagccccccgagattgcacatggctatgtggagcactcggttcgctaccagtgtaagaactactacaaactgcgcacagaaggagatggagtgtacaccttaaacaatgagaagcagtggataaataaggctgttggagataaacttcctgaatgtgaagcagtatgtgggaagcccaagaatccggcaaacccagtgcagcggatcctgggtggacacctggatgccaaaggcagctttccctggcaggctaagatggtttcccaccataatctcaccacaggtgccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggttgttctacaccctaactactcccaggtagatattgggctcatcaaactcaaacagaaggtgtctgttaatgagagagtgatgcccatctgcctaccttcaaaggattatgcagaagtagggcgtgtgggttatgtttctggctgggggcgaaatgccaattttaaatttactgaccatctgaagtatgtcatgctgcctgtggctgaccaagaccaatgcataaggcattatgaaggcagcacagtccctgaaaagaagacaccgaagagccctgtaggggtgcagcccatactgaatgaacacaccttctgtgctggcatgtctaagtaccaagaagacacctgctatggcgatgcgggcagtgcctttgccgttcacgacctggaggaggacacctggtatgcgactgggatcttaagctttgataagagctgtgctgtggctgagtatggtgtgtatgtgaaggtgacttccatccaggactgggttcagaagaccatagctgagaactaatgcaaggctggccggaagcccttgcctgaaagcaagatttcagcctggaagagggcaaagtggacgggagtggacaggagtggatgcgataagatgtggtttgaagctgatgggtgccat

SEQ ID NO: 23 - protein, 390 aa, Homo sapiens
MNSLSEANTKFMFDLFQQFRKSKENNIFYSPISITSALGMVLLGAKDNTAQQIKKVLHFDQVTENTTGKAATYHVDRSGNVHHQFQKLLTEFNKSTDAYELKIANKLFGEKTYLFLQEYLDAIKKFYQTSVESVDFANAPEESRKKINSWVESQTNEKIKNLIPEGNIGSNTTLVLVNAIYFKGQWEKKFNKEDTKEEKFWPNKNTYKSIQMMRQYTSFHFASLEDVQAKVLEIPYKGKDLSMIVLLPNEIDGLQKLEEKLTAEKLMEWTSLQNMRETRVDLHLPRFKVEESYDLKDTLRTMGMVDIFNGDADLSGMTGSRGLVLSGVLHKAFVEVTEEGAEAAAATAVVGFGSSPTSTNEEFHCNHPFLFFIRQNKTNSILFYGRFSSP

SEQ ID NO: 24 - DNA, 1735 bp, Homo sapiens
agacacacatagcctctctgcccacctctgcttcctctaggaacacaggagttccagatcacatcgagttcaccatgaattcactcagtgaagccaacaccaagttcatgttcgacctgttccaacagttcagaaaatcaaaagagaacaacatcttctattcccctatcagcatcacatcagcattagggatggtcctcttaggagccaaagacaacactgcacaacagattaagaaggttcttcactttgatcaagtcacagagaacaccacaggaaaagctgcaacatatcatgttgataggtcaggaaatgttcatcaccagtttcaaaagcttctgactgaattcaacaaatccactgatgcatatgagctgaagatcgccaacaagctcttcggagaaaaaacgtatctatttttacaggaatatttagatgccatcaagaaattttaccagaccagtgtggaatctgttgattttgcaaatgctccagaagaaagtcgaaagaagattaactcctgggtggaaagtcaaacgaatgaaaaaattaaaaacctaattcctgaaggtaatattggcagcaataccacattggttcttgtgaacgcaatctatttcaaagggcagtgggagaagaaatttaataaagaagatactaaagaggaaaaattttggccaaacaagaatacatacaagtccatacagatgatgaggcaatacacatcttttcattttgcctcgctggaggatgtacaggccaaggtcctggaaataccatacaaaggcaaagatctaagcatgattgtgttgctgccaaatgaaatcgatggtctccagaagcttgaagagaaactcactgctgagaaattgatggaatggacaagtttgcagaatatgagagagacacgtgtcgatttacacttacctcggttcaaagtggaagagagctatgacctcaaggacacgttgagaaccatgggaatggtggatatcttcaatggggatgcagacctctcaggcatgaccgggagccgcggtctcgtgctatctggagtcctacacaaggcctttgtggaggttacagaggagggagcagaagctgcagctgccaccgctgtagtaggattcggatcatcacctacttcaactaatgaagagttccattgtaatcaccctttcctattcttcataaggcaaaataagaccaacagcatcctcttctatggcagattctcatccccgtagatgcaattagtctgtcactccatttggaaaatgttcacctgcagatgttctggtaaactgattgctggcaacaacagattctcttggctcatatttcttttctttctcatcttgatgatgatcgtcgtcatcaagaatttaatgattaaaatagcatgcctttctctctttctcttaataagcccacatataaatgtactttttcttccagaaaaattctccttgaggaaaaatgtccaaaataagatgaatcacttaataccgtatcttctaaatttgaaatataattctgtttgtgacctgttttaaatgaaccaaaccaaatcatactttttctttgaatttagcaacctagaaacacacatttctttgaatttaggtgatacctaaatccttcttatgtttctaaattttgtgattctataaaacacatcatcaataaaatagtgacataaaaccaaaaaaaaaaaaaaaaaaaaaaaaaaaaa

SEQ ID NO: 25 - protein, 858 aa, Homo sapiens
MVNFTVDQIRAIMDKKANIRNMSVIAHVDHGKSTLTDSLVCKAGIIASARAGETRFTDTRKDEQERCITIKSTAISLFYELSENDLNFIKQSKDGAGFLINLIDSPGHVDFSSEVTAALRVTDGALVVVDCVSGVCVQTETVLRQAIAERIKPVLMMNKMDRALLELQLEPEELYQTFQRIVENVNVIISTYGEGESGPMGNIMIDPVLGTVGFGSGLHGWAFTLKQFAEMYVAKFAAKGEGQLGPAERAKKVEDMMKKLWGDRYFDPANGKFSKSATSPEGKKLPRTFCQLILDPIFKVFDAIMNFKKEETAKLIEKLDIKLDSEDKDKEGKPLLKAVMRRWLPAGDALLQMITIHLPSPVTAQKYRCELLYEGPPDDEAAMGIKSCDPKGPLMMYISKMVPTSDKGRFYAFGRVFSGLVSTGLKVRIMGPNYTPGKKEDLYLKPIQRTILMMGRYVEPIEDVPCGNIVGLVGVDQFLVKTGTITTFEHAHNMRVMKFSVSPVVRVAVEAKNPADLPKLVEGLKRLAKSDPMVQCIIEESGEHIIAGAGELHLEICLKDLEEDHACIPIKKSDPVVSYRETVSEESNVLCLSKSPNKHNRLYMKARPFPDGLAEDIDKGEVSARQELKQRARYLAEKYEWDVAEARKIWCFGPDGTGPNILTDITKGVQYLNEIKDSVVAGFQWATKEGALCEENMRGVRFDVHDVTLHADAIHRGGGQIIPTARRCLYASVLTAQPRLMEPIYLVEIQCPEQVVGGIYGVLNRKRGHVFEESQVAGTPMFVVKAYLPVNESFGFTADLRSNTGGQAFPQCVFDHWQILPGDPFDNSSRPSQVVAETRKRKGLKEGIPALDNFLDKL

SEQ ID NO: 26 - DNA, 2915 bp, Homo sapiens
gcgactcgcttctttcggttctacctgggagaatccaccgccatccgccaccatggtgaacttcacggtagaccagatccgcgccatcatggacaagaaggccaacatccgcaacatgtctgtcatcgcccacgtggaccatggcaagtccacgctgacagactccctggtgtgcaaggcgggcatcatcgcctcggcccgggccggggagacacgcttcactgatacccggaaggacgagcaggagcgttgcatcaccatcaagtcaactgccatctccctcttctacgagctctcggagaatgacttgaacttcatcaagcagagcaaggacggtgccggcttcctcatcaacctcattgactcccccgggcatgtcgacttctcctcggaggtgactgctgccctccgagtcaccgatggcgcattggtggtggtggactgcgtgtcaggcgtgtgcgtgcagacggagacagtgctgcggcaggccattgccgagcgcatcaagcctgtgctgatgatgaacaagatggaccgcgccctgctggagctgcagctggagcccgaggagctctaccagactttccagcgcatcgtggagaacgtgaacgtcatcatctccacctacggcgagggcgagagcggccccatgggcaacatcatgatcgatcctgtcctcggtaccgtgggctttgggtctggcctccacgggtgggccttcaccctgaagcagtttgccgagatgtatgtggccaagttcgccgccaagggggagggccagttggggcctgccgagcgggccaagaaagtagaggacatgatgaagaagctgtggggtgacaggtactttgacccagccaacggcaagttcagcaagtcagccaccagccccgaagggaagaagctgccacgcaccttctgccagctgatcctggaccccatcttcaaggtgtttgatgcgatcatgaatttcaagaaagaggagacagcaaaactgatagagaaactggacatcaaactggacagcgaggacaaggacaaagaaggcaaacccctgctgaaggctgtgatgcgccgctggctgcctgccggagacgccttgttgcagatgatcaccatccacctgccctcccctgtgacggcccagaagtaccgctgcgagctcctgtacgaggggcccccggacgacgaggctgccatgggcattaaaagctgtgaccccaaaggccctcttatgatgtatatttccaaaatggtgccaacctccgacaaaggtcggttctacgcctttggacgagtcttctcggggctggtctccactggcctgaaggtcaggatcatggggcccaactatacccctgggaagaaggaggacctctacctgaagccaatccagagaacaatcttgatgatgggccgctacgtggagcccatcgaggatgtgccttgtgggaacattgtgggcctcgtgggcgtggaccagttcctggtgaagacgggcaccatcaccaccttcgagcacgcgcacaacatgcgggtgatgaagttcagcgtcagccctgttgtcagagtggccgtggaggccaagaacccggctgacctgcccaagctggtggaggggctgaagcggctggccaagtccgaccccatggtgcagtgcatcatcgaggagtcgggagagcacatcatcgcgggcgccggcgagctgcacctggagatctgcctgaaggacctggaggaggaccacgcctgcatccccatcaagaaatctgacccggtcgtctcgtaccgcgagacggtcagtgaagagtcgaacgtgctctgcctctccaagtcccccaacaagcacaaccggctgtacatgaaggcgcggcccttccccgacggcctggccgaggacatcgataaaggcgaggtgtccgcccgtcaggagctcaagcagcgggcgcgctacctggccgagaagtacgagtgggacgtggctgaggcccgcaagatctggtgctttgggcccgacggcaccggccccaacatcctcaccgacatcaccaagggtgtgcagtacctcaacgagatcaaggacagtgtggtggccggcttccagtgggccaccaaggagggcgcactgtgtgaggagaacatgcggggtgtgcgcttcgacgtccacgacgtcaccctgcacgccgacgccatccaccgcggagggggccagatcatccccacagcacggcgctgcctctacgccagtgtgctgaccgcccagccacgcctcatggagcccatctaccttgtggagatccagtgtccagagcaggtggtcggtggcatctacggggttttgaacaggaagcggggccacgtgttcgaggagtcccaggtggccggcacccccatgtttgtggtcaaggcctatctgcccgtcaacgagtcctttggcttcaccgctgacctgaggtccaacacgggcggccaggcgttcccccagtgtgtgtttgaccactggcagatcctgcccggagaccccttcgacaacagcagccgccccagccaggtggtggcggagacccgcaagcgcaagggcctgaaagaaggcatccctgccctggacaacttcctggacaaattgtaggcggcccttcctgcagcgcctgccgccccggggactcgcagcacccacagcaccacgtcctcgaattctcagacgacacctggagactgtcccgacacagcgacgctcccctgagaggtttctggggcccgctgcgtgccatcactcaaccataacactttttccatttcttcagagggaaactcagatgtccaaactaattttaacaaacgcattaagaggtttatttgggtacatggcccgcagtggcttttgccccagaaaggggaaaggaacacgcgggtag

SEQ ID NO: 27 - protein, 412 aa, Homo sapiens
MQPSSLLPLALCLLAAPASALVRIPLHKFTSIRRTMSEVGGSVEDLIAKGPVSKYSQAVPAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAARL

SEQ ID NO: 28 - DNA, 1239 bp, Homo sapiens
atgcagccctccagccttctgccgctcgccctctgcctgctggctgcacccgcctccgcgctcgtcaggatcccgctgcacaagttcacgtccatccgccggaccatgtcggaggttgggggctctgtggaggacctgattgccaaaggccccgtctcaaagtactcccaggcggtgccagccgtgaccgaggggcccattcccgaggtgctcaagaactacatggacgcccagtactacggggagattggcatcgggacgcccccccagtgcttcacagtcgtcttcgacacgggctcctccaacctgtgggtcccctccatccactgcaaactgctggacatcgcttgctggatccaccacaagtacaacagcgacaagtccagcacctacgtgaagaatggtacctcgtttgacatccactatggctcgggcagcctctccgggtacctgagccaggacactgtgtcggtgccctgccagtcagcgtcgtcagcctctgccctgggcggtgtcaaagtggagaggcaggtctttggggaggccaccaagcagccaggcatcaccttcatcgcagccaagttcgatggcatcctgggcatggcctacccccgcatctccgtcaacaacgtgctgcccgtcttcgacaacctgatgcagcagaagctggtggaccagaacatcttctccttctacctgagcagggacccagatgcgcagcctgggggtgagctgatgctgggtggcacagactccaagtattacaagggttctctgtcctacctgaatgtcacccgcaaggcctactggcaggtccacctggaccaggtggaggtggccagcgggctgaccctgtgcaaggagggctgtgaggccattgtggacacaggcacttccctcatggtgggcccggtggatgaggtgcgcgagctgcagaaggccatcggggccgtgccgctgattcagggcgagtacatgatcccctgtgagaaggtgtccaccctgcccgcgatcacactgaagctgggaggcaaaggctacaagctgtccccagaggactacacgctcaaggtgtcgcaggccgggaagaccctctgcctgagcggcttcatgggcatggacatcccgccacccagcgggccactctggatcctgggcgacgtcttcatcggccgctactacactgtgtttgaccgtgacaacaacagggtgggcttcgccgaggctgcccgcctttaa

(19) United States
(12) Patent Application Publication
(10) Pub. No.: US 2017/0097352 A1
(43) Pub. Date: Apr. 6, 2017

(54) IMMUNOGLOBULIN-BOUND EXTRACELLULAR VESICLES AND USES THEREOF

(71) Applicant: University of Louisville Research Foundation, Inc., Louisville, KY (US)

(72) Inventors: Douglas D. Taylor, Monmouth Junction, NJ (US); Cicek Gercel-Taylor, Monmouth Junction, NJ (US)

(21) Appl. No.: 15/152,901

(22) Filed: May 12, 2016

Related U.S. Application Data
(63) Continuation of application No. 13/795,708, filed on Mar. 12, 2013, now abandoned.
(60) Provisional application No. 61/747,632, filed on Dec. 31, 2012.

(51) Int. Cl. G01N 33/574; G01N 33/68

(52) U.S. Cl. CPC G01N 33/574; G01N 33/6854

(57) ABSTRACT

Provided are methods of isolating IgG-bound (e.g., IgG-bound or protein G-recognized IgG-bound) extracellular vesicles from a sample containing a biological fluid from a subject, where the IgG (e.g., IgG₂ or protein G-recognized IgG) is bound to an antigen present on the surface of the extracellular vesicle and the IgG (e.g., IgG₂ or protein G-recognized IgG) is an endogenous antibody. Also provided are methods of diagnosing a cancer in a subject that include detecting the presence of one or more tumor antigens in an isolated IgG-bound (e.g., IgG₂-bound or protein G-recognized IgG-bound) extracellular vesicle, and methods of treating a subject that include administering one or more cancer therapeutics to a subject having an IgG-bound (e.g., IgG₂-bound or protein G-recognized IgG-bound) extracellular vesicle that contains one or more tumor antigens.

### CROSS-REFERENCE TO RELATED APPLICATIONS

This application is a continuation of U.S. patent application Ser. No. 13/795,708, filed Mar. 12, 2013, which claims priority to U.S. Provisional Patent Application Ser. No. 61/747,632, filed Dec. 31, 2012, the entire contents of each of which are incorporated by reference herein.

### TECHNICAL FIELD

This invention relates to methods of diagnosing and treating cancer through the isolation of IgG-bound (e.g., IgG₂-bound or protein G-recognized IgG-bound) extracellular vesicles and the identification of biomarkers of cancer in these isolated extracellular vesicles.

### BACKGROUND

Cancer is associated with the induction of humoral immunity characterized by the generation of reactive IgG against a wide range of tumor antigens (Th2 response). The mechanisms underlying the induction of a humoral immune response to autologous protein antigens are multifaceted and include, for example, mutations, changes in post-translational modification(s), overexpression, ectopic expression, subcellular compartment translocations, splice variant products, or errors in proteolytic processing of certain proteins. Autoantibody responses to antigens broadly expressed in normal and cancer tissues appears to be attributable to tumor-specific mutations or post-translational modifications in proteins.

### SUMMARY

The present invention is based, at least in part, on the discovery that IgG-bound (e.g., IgG₂-bound or protein G-recognized IgG-bound) extracellular vesicles in cancer patients contain multiple tumor antigens. Thus, the present invention includes methods for isolating an IgG-bound (e.g., IgG₂-bound or protein G-recognized IgG-bound) extracellular vesicle, and methods of diagnosing and/or differentiating a cancer in a subject and selecting a subject for participation in a clinical study that include isolating an IgG-bound (e.g., IgG₂-bound or protein G-recognized IgG-bound) extracellular vesicle from a sample containing biological fluid from the subject. Also provided are methods of treating a subject that include selectively administering one or more cancer therapeutics to a subject that has been determined to have cancer based on the presence of IgG-bound (e.g., IgG₂-bound or protein G-recognized IgG-bound) extracellular vesicles that contain one or more tumor antigens.

Provided herein are methods of isolating an IgG₂-bound extracellular vesicle, wherein the IgG₂ is bound to an antigen present on the surface of the extacellular vesicle and the IgG₂ is an endogenous antibody, that include (a) obtaining a sample comprising a biological fluid from the subject, (b) contacting the sample with an agent that specifically binds to a heavy chain constant domain of IgG₂, (c) separating the agent bound with IgG₂-bound extracellular vesicles from unbound material present in the sample, and (d) dissociating the IgG₂-bound extracellular vesicles from the agent, thereby isolating IgG₂-bound extracellular vesicles.

Also provided herein are methods of isolating a protein G-recognized IgG-bound extracellular vesicle, where the protein G-recognized IgG is bound to an antigen present on the surface of the extracellular vesicle and the protein G-recognized IgG is an endogenous antibody, that include: (a) obtaining a sample comprising a biological fluid from the subject, (b) contacting the sample with an agent that specifically binds to a heavy chain constant domain of one or more protein G-recognized IgG, (c) separating the agent bound with protein G-recognized IgG-bound extracellular vesicles from unbound material present in the sample, and (d) dissociating the protein G-recognized IgG-bound extracellular vesicles from the agent, thereby isolating protein G-recognized IgG-bound extracellular vesicles. In some embodiments of any of the methods described herein, the protein G-recognized IgG is IgG₂. In some embodiments of any of the methods described herein, the agent is protein G. In some embodiments of any of the methods described herein, the agent is an antibody that specifically binds to a heavy chain constant domain of IgG₂.

In some embodiments of any of the methods described herein, the biological fluid is blood, serum, plasma, urine, cerebrospinal fluid, saliva, tears, nasal discharge, semen, amniotic fluid, vaginal discharge, lymph, tears, mucus, synovial fluid, breast milk, vitreous humor, aqueous humor, or sweat. Some embodiments of any of the methods described herein further include, before the contacting in (b), enriching the sample for extracellular vesicles by passing the sample through a molecular sieve column or a molecular weight filter. Some embodiments of any of the methods described herein further include (e) centrifuging the eluate to generate a pellet, and (f) resuspending the pellet in a physiologically acceptable buffer. In some embodiments of any of the methods described herein, the sample is obtained from a subject having or suspected of having a cancer (e.g., bladder cancer, epithelial cancer, prostate cancer, anal cancer, appendix cancer, bone cancer, brain tumor, breast cancer, heart cancer, cervical cancer, colon cancer, gallbladder cancer, stomach cancer, head and neck cancer, liver cancer, kidney cancer, laryngeal cancer, lung cancer, ovarian cancer, pancreatic cancer, penile cancer, pituitary cancer, rectal cancer, salivary gland cancer, sarcoma, testicular cancer, throat cancer, thyroid cancer, urethral cancer, uterine cancer, vaginal cancer, or vulvar cancer). In some embodiments of any of the methods described herein, the subject is human.

In some embodiments of any of the methods described herein, the protein G-recognized IgG-bound extracellular vesicle or IgG₂-bound extracellular vesicle contains at least one tumor antigen. In some embodiments of any of the methods described herein, the at least one tumor antigen is selected from the group of: epidermal growth factor receptor (EGFR), complement 7, cystatin-A, alpha-1-antitrypsin, monocyte/macrophage Ig-related receptor-10, annexin A1, annexin A2, arginase-1, complement component C4B-1, haptoglobin, serpin peptidase inhibitor, clade B (Ovalbumin), member 3, eukaryotic translation elongation factor 2, and cathepsin D.

Also provided are methods of diagnosing a cancer in a subject that include: (a) obtaining a sample comprising a biological fluid from the subject; (b) isolating an IgG₂-bound extracellular vesicle from the sample, where the IgG₂-bound extracellular vesicle contains an IgG₂ bound to an antigen present on the surface of the extracellular vesicle and the IgG₂ bound to the extracellular vesicle is an endogenous antibody; and (c) detecting the presence of one or more tumor antigens in the isolated IgG₂-bound extracellular vesicle; and (d) diagnosing a subject having an isolated IgG₂-bound extracellular vesicle containing one or more tumor antigens as having a cancer.

Also provided herein are methods of diagnosing a cancer in a subject that include: (a) obtaining a sample comprising a biological fluid from the subject; (b) isolating a protein G-recognized IgG-bound extracellular vesicle from the sample, where the protein G-recognized IgG-bound extracellular vesicle contains a protein G-recognized IgG bound to an antigen present on the surface of the extracellular vesicle and the protein G-recognized IgG bound to the extracellular vesicle is an endogenous antibody; and (c) detecting the presence of one or more tumor antigens in the isolated protein G-recognized IgG-bound extracellular vesicle; and (d) diagnosing a subject having an isolated protein G-recognized IgG-bound extracellular vesicle containing one or more tumor antigens as having a cancer. In some embodiments of any of the methods described herein, the protein G-regonized IgG is IgG₂. In some embodiments of any of the methods described herein, the biological fluid is blood, serum, plasma, urine, cerebrospinal fluid, saliva, tears, nasal discharge, semen, amniotic fluid, vaginal discharge, lymph, tears, mucus, synovial fluid, breast milk, vitreous humor, aqueous humor, or sweat.

In some mebodiments of any of the methods described herein, in (d), a subject having a protein G-recognized-IgG-bound extracellular vesicle or IgG₂-bound extracellular vesicle containing one of more tumor antigens selected from the group of: epidermal growth factor receptor (EGFR), complement 7, cystatin-A, alpha-1-antitrypsin, monocyte/macrophage Ig-related receptor-10, annexin A1, annexin A2, arginase-1, complement component C4B-1, haptoglobin, serpin peptidase inhibitor, clade B (Ovalbumin), member 3, eukaryotic translation elongation factor 2, and cathepsin D is diagnosed with ovarian cancer. In some embodiments of any of the methods described herein, the isolating in (b) includes contacting the sample with an agent that specifically binds to a heavy chain constant domain of IgG₂ (e.g., protein G or an antibody that specifically binds to the heavy chain constant domain of IgG₂). In some embodiments of any of the methods described herein, the detecting in (c) is performed using mass spectrometry and/or using two-dimensional gel electrophoresis. In some embodiments of any of the methods described herein, the detecting in (c) is performed using an exogenous antibody that specifically binds to a tumor antigen.

In some embodiments of any of the methods described herein the subject is a human. Some embodiments of any of the methods described herein further include administering to the subject diagnosed with cancer one or more cancer therapeutics.

Also provided herein are methods of treating a subject that include selectively administering one or more cancer therapeutics to a subject that has been determined to have an IgG₂-bound extracellular vesicle that contains one or more tumor antigens, wherein the IgG₂-bound extracellular vesicle contains an IgG₂ bound to an antigen present on the surface of the extracellular vesicle and the IgG₂ bound to the extracellular vesicle is an endogenous antibody.

Also provided are methods of treating a subject that include selectively administering one or more cancer therapeutics to a subject that has been determined to have a protein G-recognized IgG-bound extracellular vesicle that contains one or more tumor antigens, wherein the protein G-recognized IgG-bound extracellular vesicle contains a protein G-recognized IgG bound to an antigen present on the surface of the extracellular vesicle and the protein G-recognized IgG bound to the extracellular vesicle is an endogenous antibody. In some embodiments of any of the methods described herein, the protein G-recognized IgG is IgG₂. In some embodiments of any of the methods described herein, the subject has been determined to have a protein G-recognized IgG-bound extracellular vesicle or an IgG₂-bound extracellular vesicle that contains one or more tumor antigens selected from the group of: epidermal growth factor receptor (EGFR), complement 7, cystatin-A, alpha-1-antitrypsin, monocyte/macrophage Ig-related receptor-10, annexin A1, annexin A2, arginase-1, complement component C4B-1, haptoglobin, serpin peptidase inhibitor, clade B (Ovalbumin), member 3, eukaryotic translation elongation factor 2, and cathepsin D.

In some embodiments of any of the methods described herein, the one or more cancer therapeutic is selected from the group of: an antimetabolite, an alkylating agent, interleukin-2, and a therapeutic antibody. In some embodiments of any of the methods described herein, the administering is performed by oral, intravenous, intraarterial, subcutaneous, intramuscular, intraperitoneal, or intrathecal administration.

Also provided herein are methods of selecting a subject for participation in a clinical trial that include: (a) obtaining a sample comprising a biological fluid from a subject; (b) isolating an IgG₂-bound extracellular vesicle from the sample, wherein the IgG₂-bound extracellular vesicle contains an IgG₂ bound to an antigen present on the surface of the extracellular vesicle and the IgG₂ bound to the extracellular vesicle is an endogenous antibody; (c) detecting the presence of one or more tumor antigens in the isolated IgG₂-bound extracellular vesicle; and (d) selecting a subject having isolated IgG₂-bound extracellular vesicles containing one or more tumor antigens for participation in a clinical study.

Also provided herein are methods of selecting a subject for participation in a clinical trial that include: (a) obtaining a sample comprising a biological fluid from a subject; (b) isolating a protein G-recognized IgG-bound extracellular vesicle from the sample, wherein the protein G-recognized IgG-bound extracellular vesicle contains a protein G-recognized IgG bound to an antigen present on the surface of the extracellular vesicle and the protein G-recognized IgG bound to the extracellular vesicle is an endogenous antibody; (c) detecting the presence of one or more tumor antigens in the isolated protein G-recognized IgG-bound extracellular vesicle; and (d) selecting a subject having isolated protein G-recognized IgG-bound extracellular vesicles containing one or more tumor antigens for participation in a clinical study. In some embodiments of any of the methods described herein, the protein G-recognized IgG is IgG₂.

Some embodiments of any of the methods described herein further include administering one or more cancer therapeutics to the subject during the clinical study. In some embodiments of any of the methods described herein, the biological fluid is blood, serum, plasma, urine, cerebrospinal fluid, saliva, tears, nasal discharge, semen, amniotic fluid, vaginal discharge, lymph, tears, mucus, synovial fluid, breast milk, vitreous humor, aqueous humor, or sweat. In some embodiments of any of the methods described herein, the isolating in (b) comprises contacting the sample with an agent that specifically binds to a heavy chain constant domain of IgG₂ (e.g., protein G or an antibody that specifically binds to the heavy chain constant domain of IgG₂).

In some embodiments of any of the methods described herein, the detecting in (c) is performed using mass spectrometry or two-dimensional gel electrophoresis. In some embodiments of any of the methods described herein, the detecting in (c) is performed using an exogenous antibody that specifically binds to a tumor antigen. In some embodiments of any of the methods described herein, the subject is a human.

As used herein, by the term “presence” is meant a level that is greater than a threshold level (e.g., a threshold detection level of an assay for determining the presence or absence of a protein or mRNA, or a level of expression (protein or mRNA) in a control subject (e.g., a subject not having or suspected of having a cancer, preferably an age-, sex-, and/or race-matched subject)). Additional threshold levels can be determined using methods described herein and known in the art.

As used herein, by the term “absence” is meant a level that is less than a threshold level (e.g., a threshold detection level of an assay for determining the presence or absence of a protein or mRNA).

By the term “isolating” is meant the enrichment of a material (e.g., IgG-bound extracellular vesicles, such as IgG₂-bound extracellular vesicles or protein G-recognized IgG-bound extracellular vesicles) from a starting material (e.g., a sample containing a biological fluid). For example, isolating an IgG-bound (e.g., IgG₂-bound or protein G-recognized IgG-bound) extracellular vesicle can include an at least 1-fold, 2-fold, 3-fold, 4-fold, or 5-fold enrichment of IgG-bound (e.g., IgG₂-bound or protein G-recognized IgG-bound) extracellular vesicles in a solution as compared to the starting material. In some embodiments, the isolating results in a solution of IgG-bound (e.g., IgG₂-bound or protein G-recognized IgG-bound) extracellular vesicles that is at least 50%, 55%, 60%, 65%, 70%, 85%, 80%, 85%, 90%, 91%, 92%, 93%, 94%, 95%, 96%, 97%, 98%, or 99% pure, by weight.

As used herein, a “subject” is a vertebrate, including any member of the class mammalia, including humans, domestic and farm animals, and zoo, sports or pet animals, such as mouse, rabbit, pig, sheep, goat, cattle, horse (e.g., race horse), and higher primates. In preferred embodiments, the subject is a human.

By the term “detecting” is meant measuring or identifying the presence of a molecule (e.g., protein or a protein fragment; or an mRNA) in a sample (e.g., a sample containing an IgG-bound (e.g., IgG₂-bound or protein G-recognized IgG-bound) extracellular vesicle). Detecting, as described herein, can include identifying or measuring the presence or absence of one or more (e.g., one, two, three, four, five, six, seven, eight, nine, ten, eleven, twelve, or thirteen) protein(s) having at least 10 (e.g., at least 11, 12, 13, 14, 15, 16, 17, 18, 19, 20, or 25) contiguous amino acids of complement 7 (C7), cystatin-A, alpha-1-antitrypsin, monocyte/macrophage Ig-related receptor-10 (MIR-10), annexin A1 (ANXA1), annexin A2 (ANXA2), arginase-1 (ARG-1), complement component C4B-1, haptoglobin, serpin peptidase inhibitor, clade B (Ovalbumin), member 3 (SERPINB3), eukaryotic translation elongation factor 2 (EEF2), cathepsin D (CTSD), and epidermal growth factor receptor (EGFR) in a sample. Exemplary proteins that can be detected contain at least 10 (e.g., at least 11, 12, 13, 14, 15, 16, 17, 18, 19, 20, or 25) contiguous amino acids of a sequence within any one of SEQ ID NOS: 1, 3, 5, 7, 9, 11, 13, 15, 17, 19, 21, 23, 25, and 27. The contiguous amino acid sequence can be present within any portion of the sequence of SEQ ID NOS: 1, 3, 5, 7, 9, 11, 13, 15, 17, 19, 21, 23, 25, and 27, for example, a sequence starting at the N-terminus, a sequence ending at the C-terminus, or a sequence starting at any single amino acid within the sequence (with the exception of the last four amino acids at the C-terminus of the protein). Exemplary proteins that can be detected are SEQ ID NO: 1, 3, 5, 7, 9, 11, 13, 15, 17, 19, 21, 23, 25, and 27.

Detecting, as used herein, can include identifying or measuring the presence or absence of one or more (e.g., one, two, three, four, five, six, seven, eight, nine, ten, eleven, twelve, or thirteen) mRNAs that contain at least 15 nucleotides (e.g., at least 20, 25, 30, 35, 40, 45, 50, 60, 70, 80, 90, or 100 nucleotides) of an mRNA encoding C7, cystatin-A, alpha-1-antitrypsin, MIR-10, ANXA1, ANXA2, ARG-1, complement component C4B-1, haptoglobin, SERPINB3, EEF2, CTSD, or EGFR in a sample. Exemplary mRNAs that can be detected contain at least 15 (e.g., at least 20, 25, 30, 35, 40, 45, 50, 60, 70, 80, 90, or 100 nucleotides) contiguous nucleotides of a sequence within any one of SEQ ID NOs: 2, 4, 6, 8, 10, 12, 14, 16, 18, 20, 22, 24, 26, and 28. The contiguous nucleotide sequence can be present within any portion of the sequence of SEQ ID NOs: 2, 4, 6, 8, 10, 12, 14, 16, 18, 20, 22, 24, 26, or 28, for example, a sequence starting at the 5′ end, a sequence ending at the 3′ end, or a sequence starting at any single nucleotide within the sequence (with the exception of the last ten nucleotides at the 5′ end of the sequence). Exemplary mRNAs that can be detected are SEQ ID NOs: 2, 4, 6, 8, 10, 12, 14, 16, 18, 20, 22, 24, 26, or 28.

By the phrase “cancer therapeutic” is meant an agent that is administered to a subject for the purpose of treating or reducing the progression of cancer in a mammal. Non-limiting examples of cancer therapeutics can include those that induce cancer cell death (e.g., cancer cell apoptosis) in a mammal. In some embodiments, a cancer therapeutic can reduce the rate of cancer cell division (e.g., reduce the rate of tumor mass growth) or tumor metastasis in a mammal (e.g., as compared to a similar subject having the same type of cancer and receiving no treatment or a different treatment). Non-limiting examples of cancer therapeutics include antimetabolites, alkylating agents, interleukin-2, and therapeutic antibodies (e.g., trastuzumab). Exemplary cancer therapeutics are described herein. Additional examples of cancer therapeutics are known in the art.

By the term “immunoglobulin G2” or “IgG₂” is an antibody molecule that contains a γ2 heavy chain. IgG₂ also has a shorter hinge region than IgG₁. The hinge region of IgG₂ generally contains 12 amino acids and four disulfide linkages. The hinge region of IgG₂ lacks a glycine residue and forms a relatively short and rigid poly-proline double helix that is stabilized by inter-heavy chain disulfide bridges.

By the term “immunoglobulin” or “IgG” is meant a mammalian IgG antibody. For example, an IgG can be IgG₁, IgG₂, IgG₃, or IgG₄.

By the term “protein G-recognized immunoglobulin” or “protein G-recognized IgG” is meant one or more mammalian IgG antibodies that contain a heavy chain constant domain that is specifically bound by protein G.

By the term “extracellular vesicle” is meant a lipid-based vesicle ranging from 20-200 nm in diameter that expresses tetraspanin (CD63) that is present in a sample (e.g., a biological fluid) obtained from a subject. The term extracellular vesicle is also referred to in the art as an exosome, microvesicle, or nanovesicle. In some embodiments, an extracellular vesicle is between about 50 nm to about 200 nm in diameter. Extracellular vesicles are secreted or shed from a variety of different mammalian cell types (e.g., cancer cells). Non-limiting examples of extracellular vesicles and methods for the enrichment of extracellular vesicles from a sample (e.g., a biological fluid) obtained from a mammalian subject are described herein. Additional examples of extracellular vesicles and methods for the enrichment of extracellular vesicles from a sample obtained from a mammalian subject are known in the art. Specific methods for isolating an IgG-bound (e.g., IgG₂-bound or protein G-recognized IgG-bound) extracellular vesicle are described herein.

By the term “IgG₂-bound extracellular vesicle” is meant an extracellular vesicle that has an endogenous IgG₂ antibody bound to an antigen that is at least partially exposed on the extracellular vesicle surface.

By the term “IgG-bound extracellular vesicle” is meant an extracellular vesicle that has an endogenous IgG (e.g., IgG₁, IgG₂, IgG₃, or IgG₄) antibody bound to an antigen that is at least partially exposed on the extracellular vesicle surface.

By the term “protein G-recognized IgG-bound extracellular vesicle” is meant an extracellular vesicle that has one or more endogenous IgG antibodies bound to an antigen that is at least partially exposed on the extracellular vesicle surface, where the one or more endogenous IgG antibodies contain a heavy chain constant domain that is specifically bound by protein G.

By the term “biological fluid” is a meant a physiological fluid obtained from a mammalian subject (e.g., a composition containing blood, serum, plasma, urine, cerebrospinal fluid, saliva, tears, nasal discharge, semen, amniotic fluid, vaginal discharge, lymph, tears, mucus, synovial fluid, breast milk, vitreous humor, aqueous humor, or sweat).

By the term “tumor antigen” is meant a molecule (e.g., a protein, lipid, a mRNA, a sugar, or a combination or sub-combination thereof) that is uniquely expressed or differently expressed (e.g., increased expression, different post-translational modification(s), different subcellular location, or different splice form) in a cancer cell as compared to a control cell (e.g., a non-cancerous cell of the same tissue type). Non-limiting examples of tumor antigens are described herein. Additional examples of tumor antigens are known in the art.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is schematic showing an exemplary method for isolating extracellular vesicle-reactive IgG (e.g., IgG₂ or protein G-recognized IgG).

FIG. 2 is a set of six graphs showing the size of IgG-bound (e.g., IgG₂-bound or protein G-recognized IgG-bound) extracellular vesicle fractions (“Bound”) (right three graphs) and IgG-unbound (e.g., IgG₂-unbound or protein G-recognized IgG-unbound) extracellular vesicle fractions (“Unbound”) (left three graphs) in a sample from an ovarian cancer subject.

FIG. 3A is a graph showing the size of total circulating extracellular vesicles from an ovarian cancer subject.

FIG. 3B is a graph showing the size of IgG-bound (e.g., IgG₂-bound or protein G-recognized IgG-bound) extracellular vesicles isolated from an ovarian cancer subject.

FIG. 4 is an immunoblot showing the presence of epidermal growth factor receptor (EGFR) in IgG-bound (e.g., IgG₂-bound or protein G-recognized IgG-bound) extracellular vesicles (“Bound”) and IgG-unbound (e.g., IgG₂-unbound or protein G-recognized IgG-unbound) (“Unbound”) extracellular vesicles from three different ovarian cancer patients (Patient A, B, or C).

FIG. 5A is a list of proteins identified in IgG-bound (e.g., IgG₂-bound or protein G-recognized IgG-bound) extracellular vesicles isolated from a first subject having ovarian cancer identified by two-dimensional gel electrophoresis and mass spectrometry. The proteins shown in bold were not detected in IgG-unbound (e.g., IgG₂-unbound or protein G-recognized IgG-unbound) extracellular vesicles from the same subject and are implicated for a role in cancer.

FIG. 5B is a summary of the mass spectrometry data from the IgG-bound (e.g., IgG₂-bound or protein G-recognized IgG-bound) extracellular vesicles isolated from the first subject having ovarian cancer.

FIG. 6A is a list of proteins identified in IgG-unbound (e.g., IgG₂-unbound or protein G-recognized IgG-unbound) extracellular vesicles isolated from a first subject having ovarian cancer (i.e., the same subject as in FIG. 5) identified by two-dimensional gel electrophoresis and mass spectrometry.

FIG. 6B is a summary of the mass spectrometry data from the IgG-unbound (e.g., IgG₂-unbound or protein G-recognized IgG-unbound) extracellular vesicles isolated from the first subject having ovarian cancer.

FIG. 7A is a list of proteins identified in IgG-bound (e.g., IgG₂-bound or protein G-recognized IgG-bound) extracellular vesicles isolated from a second subject having ovarian cancer identified by two-dimensional gel electrophoresis and mass spectrometry.

FIG. 7B is a summary of the mass spectrometry data from the IgG-bound (e.g., IgG₂-bound or protein G-recognized IgG-bound) extracellular vesicles isolated from the second subject having ovarian cancer.

FIG. 8A is a list of proteins identified in IgG-unbound (e.g., IgG₂-unbound or protein G-recognized IgG-unbound) extracellular vesicles isolated from a second subject having ovarian cancer (i.e., the same subject as in FIG. 7) identified by two-dimensional gel electrophoresis and mass spectrometry.

FIG. 8B is a summary of the mass spectrometry data from the IgG-unbound (e.g., IgG₂-unbound or protein G-recognized IgG-unbound) extracellular vesicles isolated from the second subject having ovarian cancer.

### DETAILED DESCRIPTION

Provided herein are methods for isolating IgG-bound (e.g., IgG₂-bound or protein G-recognized IgG-bound) extracellular vesicles. These methods include obtaining a sample containing a biological fluid from a subject, contacting the sample with an agent that specifically binds to a heavy chain constant domain of an IgG (e.g., IgG₂ or protein G-recognized IgG), separating the agent bound with IgG-bound (e.g., IgG₂-bound or protein G-recognized IgG-bound) extracellular vesicles from unbound material present in the sample, and dissociating the IgG-bound (e.g., IgG₂-bound or protein G-recognized IgG-bound) extracellular vesicles from unbound material present in the sample. Also provided are methods of diagnosing a cancer, treating a subject, and selecting a subject for participation in a clinical trial that include isolating an IgG-bound (e.g., IgG₂-bound or protein G-recognized IgG-bound) extracellular vesicle from a sample containing a biological fluid from a subject.

Various aspects of these methods are described herein. Any one or more of these various aspects can be combined in any manner without limitation.

### Methods of Isolating IgG-Bound Extracellular Vesicles

Provided herein are methods of isolating an IgG-bound (e.g., IgG₂-bound or protein G-recognized IgG-bound) extracellular vesicle, where the IgG (e.g., IgG₂ or protein G-recognized IgG) is bound to an antigen present on the surface of the extracellular vesicle and the IgG (e.g., IgG₂ or protein G-recognized IgG) is an endogenous antibody. These methods include obtaining a sample containing a biological fluid from the subject, contacting the sample with an agent that specifically binds to a heavy chain constant domain of IgG (e.g., IgG₂ or protein G-recognized IgG), separating the agent bound with IgG-bound (e.g., IgG₂-bound or protein G-recognized IgG-bound) extracellular vesicles from unbound material present in the sample, and dissociating the IgG-bound (e.g., IgG₂-bound or protein G-recognized IgG-bound) extracellular vesicles from the agent, thereby isolating the IgG-bound (e.g., IgG₂-bound or protein G-recognized IgG-bound) extracellular vesicles.

In some embodiments, the sample containing a biological fluid can contain serum, plasma, urine, cerebrospinal fluid, saliva, tears, nasal discharge, semen, amniotic fluid, vaginal discharge, lymph, tears, mucus, synovial fluid, breast milk, vitreous humor, aqueous humor, or sweat. In some embodiments, the sample containing a biological fluid from the subject can be stored (e.g., at a temperature below 15° C., below 10° C., below 0° C., below −20° C., below −50° C., or below −70° C.) for a period of time (e.g., at least 1 hour, 12 hours, 24 hours, 36 hours, or 48 hours) prior contacting the sample with an agent that specifically binds to a heavy chain constant domain of IgG (e.g., IgG₂ or protein G-recognized IgG). In some embodiments, the sample is treated prior to the step of contacting with an agent that specifically binds to a heavy chain constant domain of IgG (e.g., IgG₂ or protein G-recognized IgG) (e.g., the sample may be centrifuged to separate cellular debris, diluted with a physiological buffer, and/or treated with anticoagulants, e.g., EDTA, sodium citrate, or heparin).

In some embodiments, the subject may be suspected of having a cancer. In some embodiments, the subject may present clinically with one or more symptoms of cancer. Non-limiting examples of cancer include fatigue, lump or thickening under the skin, weight changes, yellowing, darkening, or redness of the skin, sores that won't heal, changes in bowel or bladder habits, persistent cough, difficulty swallowing, hoarseness, persistent indigestion or discomfort after eating, persistent, unexplained muscle or joint pain, persistent fevers or night sweats, and pain. In some embodiments, the subject is identified as being at risk of developing a cancer (e.g., identified as having an increased risk of cancer based on a familial history of cancer, genetic risk, and/or environmental exposure to a toxic and/or mutagenic substance). In some embodiments, the subject is not suspected of having cancer and does not present with one or more symptoms of a cancer. In some embodiments, the subject is not considered as being at significant risk of developing a cancer. In some embodiments, the sample is obtained from a male. In some embodiments, the sample is obtained from a female. In some embodiments, the sample is obtained from a child (e.g., a child between the ages of 1 and 5, between the ages of 5 and 10, or between the ages of 10 and 18). In some embodiments, the sample is obtained from an adult (e.g., a person who is at least 18, 25, 30, 35, 40, 45, 50, 55, 60, 65, 70, 75, 80, 85, 90, 95, or 100 years old).

In some embodiments, the methods of isolation described herein are performed at periodic intervals using samples obtained from the same subject (e.g., samples obtained from the same subject at least once every month, once every two months, once every four months, once every six months, once every year, or once every two years).

Non-limiting exemplary agents that specifically bind to a heavy chain constant domain of IgG include protein A or protein L. Non-limiting exemplary agents that specifically bind to a heavy chain constant domain of IgG₂ include protein G or an antibody that specifically binds to the heavy chain constant domain of IgG₂ (e.g., a polyclonal or monoclonal antibody, e.g., mouse anti-human IgG₂ antibodies available from Invitrogen Inc. (Clone HP-6014) and BD Pharmingen (Clone G18-21)). Additional exemplary antibodies that specifically bind to the heavy chain constant domain of IgG (e.g., IgG₂ or protein G-recognized IgG) are known in the art. In some embodiments, the agent that specifically binds to a heavy chain constant domain of IgG (e.g., IgG₂ or protein G-recognized IgG) is present on the surface of a bead (e.g., a microbead or nanoparticle) such that the agent can specifically bind to the heavy chain constant domain of IgG (e.g., IgG₂ or protein G-recognized IgG). In some embodiments, the agent that specifically binds to a heavy chain constant domain of IgG (e.g., IgG₂ or protein G-recognized IgG) is present on the surface of a bead (e.g., a microbead or nanoparticle) present in a column. In some embodiments, the agent that specifically binds to a heavy chain constant domain of IgG (e.g., IgG₂ or protein G-recognized IgG) is present on a solid surface (e.g., a surface of a chip, a well, a tube, or a microfluidic or nanofluidic chamber) such that the agent can specifically bind to the heavy chain constant domain of IgG (e.g., IgG₂ or protein G-recognized IgG). In some embodiments, the agent that specifically binds to a heavy chain constant domain of IgG (e.g., IgG₂ or protein G-recognized IgG) contains a label that allows for detection or separation of the IgG-bound (e.g., IgG₂-bound or protein G-recognized IgG-bound) extracellular vesicles (e.g., a fluorophore or poly-His tag).

In some embodiments, the contacting of the sample with the agent that specifically binds to the heavy chain constant domain of IgG (e.g., IgG₂ or protein G-recognized IgG) occurs through mixing or adding the agent directly to the sample. In some embodiments, the contacting of the sample with the agent that specifically binds to the heavy chain constant domain of IgG (e.g., IgG₂ or protein G-recognized IgG) occurs through mixing or adding to the sample a bead (e.g., a microbead or nanobead) (e.g., a magnetic bead) that has attached to its surface the agent that specifically binds to the heavy chain constant domain of IgG (e.g., IgG₂ or protein G-recognized IgG), such that the agent is capable of specifically binding to the heavy chain constant domain of IgG (e.g., IgG₂ or protein G-recognized IgG). For example, the contacting with such a bead can take place as a step in a chromatographic- or bead-assisted (affinity) isolation method (e.g., fluorescence-assisted sorting, nickel column purification, or magnetic field sorting). In some embodiments, the contacting of the sample with the agent occurs through adding the sample to a surface (e.g., a surface of a chip, a well, a tube, or a microfluidic or nanofluidic chamber) that has an agent that specifically binds to the heavy chain constant domain of IgG (e.g., IgG₂ or protein G-recognized IgG) present on its surface, such that that agent can specifically bind to the heavy chain constant domain of IgG (e.g., IgG₂ or protein G-recognized IgG) present on its surface.

The contacting can be performed within any period of time suitable to allow the agent that specifically binds to a heavy chain constant domain of IgG (e.g., IgG₂ or protein G-recognized IgG) to bind to the heavy chain constant domain of IgG (e.g., IgG₂ or protein G-recognized IgG) (e.g., performed within 30 seconds to 24 hours, e.g., within 1 minute to 12 hours, within 1 minute to 6 hours, or within 1 minute to 4 hours). The contacting can be performed at any temperature that does not significantly negatively impact the integrity of the extracellular vesicles present in the sample (e.g., a temperature of between about 10° C. to about 25° C.). In some embodiments, the contacting can include agitation of the sample, such that the agent that specifically binds to a heavy chain constant domain of IgG (e.g., IgG₂ or protein G-recognized IgG) is admixed with the sample.

The agent bound with IgG-bound (e.g., IgG₂-bound or protein G-recognized IgG-bound) extracellular vesicles can be separated from the unbound material using a variety of different methods known in the art. In some embodiments, where the agent that specifically binds to the heavy chain constant domain of IgG (e.g., IgG₂ or protein G-recognized IgG) is present on the surface of a bead (e.g., a microbead or nanobead), such that the agent is capable of binding to the heavy chain constant domain of IgG (e.g., IgG₂ or protein G-recognized IgG), the agent bound with IgG-bound (e.g., IgG₂-bound or protein G-recognized IgG-bound) extracellular vesicles can be separated from the unbound material by, e.g., gravity flow with a low salt or physiological buffer when the beads are present in a column, by centrifugation and aspiration of the supernatant (with the agent bound to IgG-bound (e.g., IgG₂-bound or protein G-recognized IgG-bound) extracellular vesicles present in the pellet), using a magnet (when the beads are magnetic beads, the agent bound to the IgG-bound (e.g., IgG₂-bound or protein G-recognized IgG) extracellular vesicles is on the surface of the magnetic beads, and the beads are attracted to the magnet), or using fluorescence-assisted sorting (e.g., when the agent that specifically binds to the heavy chain constant domain of IgG (e.g., IgG₂ or protein G-recognized IgG) is labeled with a fluorophore).

In some embodiments, where the agent that specifically binds to the heavy chain constant domain of IgG (e.g., IgG₂ or protein G-recognized IgG) is present on a surface (e.g., a surface of a chip, a well, a tube, a bead, a column, or a microfluidic or nanofluidic chamber), such that the agent is capable of binding to the heavy chain constant domain of IgG (e.g., IgG₂ or protein G-recognized IgG), the agent bound with IgG-bound (e.g., IgG₂-bound or protein G-recognized IgG-bound) extracellular vesicles can be separated from the unbound material by, e.g., by washing the surface with a low salt or physiological buffer and aspirating or removing the wash solution from the surface bound with IgG-bound (e.g., IgG₂-bound or protein G-recognized IgG-bound) extracellular vesicles.

The IgG-bound (e.g., IgG₂-bound or protein G-recognized IgG-bound) extracellular vesicles can be separated from the agent using a variety of different methods including, for example, the use of a buffer that significantly decreases the ability (e.g., affinity) of the agent to bind to the heavy chain constant domain of IgG (e.g., IgG₂ or protein G-recognized IgG) (e.g., a high salt buffer). A variety of buffers that can be used to dissociate agents that specifically bind to a heavy chain constant domain of IgG (e.g., IgG₂ or protein G-recognized IgG) are known in the art. In some embodiments, the IgG-bound (e.g., IgG₂-bound or protein G-recognized IgG-bound) extracellular vesicles can be separated from the agent by using an antibody that competes with the agent for binding to a heavy chain constant domain of IgG (e.g., IgG₂ or protein G-recognized IgG).

Any of the methods described herein can further include selectively isolating or enriching the presence of IgG₂-bound extracellular vesicles. Such enrichment can be performed using the exemplary agents that selectively bind to the heavy chain constant domain of IgG₂, and using any combination of the contacting and separating steps described above.

Any of the methods described herein can further include enriching the sample for extracellular vesicles prior to contacting the sample with an agent that specifically binds to a heavy chain constant domain of IgG (e.g., IgG₂ or protein G-recognized IgG). A sample that is enriched in extracellular vesicles need not be 100% pure extracellular vesicles.

A sample can be enriched for extracellular vesicles using any methods known in the art (see, for example the techniques described in Taylor et al., Serum/Plasma Proteomics, Chapter 15, “Exosome Isolation for Proteomic Analyses and RNA Profiling,” Springer Science, 2011, and references cited therein). In some embodiments, a sample containing plasma can be enriched for extracellular vesicles using centrifugation, sodium heparin (1,000 m/L) can be added prior to enrichment and the blood can be centrifuged at 12,000×g for 15 min at 4° C. to remove any cellular debris. The cell-free blood specimens can further be centrifuged at 100,000×g for 1 h at 4° C. The pellet containing extracellular vesicles can be resuspended in phosphate buffered saline (PBS), and recentrifuged at 100,000×g for 1 h at 4° C. The resulting pelleted extracellular vesicles can be resuspended in PBS and used to isolate an IgG-bound (e.g., IgG₂-bound or protein G-recognized IgG-bound) extracellular vesicles (e.g., using any of the methods described herein).

In some embodiments, a sample can be enriched for extracellular vesicles using a size exclusion chromatography (also known as a molecular sieve chromatography). The void volume fractions can be pooled and centrifuged at 100,000×g for 1 hour at 4° C., and the resulting pellet (containing extracellular vesicles) can be resuspended and used to isolate an IgG-bound (e.g., IgG₂-bound or protein G-recognized IgG-bound) extracellular vesicles (e.g., using any of the methods described herein).

In some embodiments, a sample can be enriched for extracellular vesicles using magnetic beads. In an exemplary method, serum can be absorbed to anti-EpCAM, or anti-EGFR antibodies coupled to magnetic microbeads. Anti-EpCAM or anti-EGFR antibodies coupled to microbeads (50 mL) can be added to the serum specimens (2 mL), mixed, and incubated on a shaker for 2 h at room temperature. Each tube is thereafter placed in the magnetic separator and fluid removed, leaving the magnetic beads and the bound extracellular vesicles attached to the side of the tube. The tube is then removed from the magnetic separator and the beads rinsed with 500 mL Tris-buffered saline (TBS), and the separation repeated. After the wash step, the tube is removed from the magnetic holder and the extracellular vesicles complexed to the beads can be eluted and used to isolate IgG-bound (e.g., IgG₂-bound or protein G-recognized IgG-bound) extracellular vesicles (e.g., using any of the methods described herein).

In some embodiments, a sample can be enriched for extracellular vesicles using precipitation. In one exemplary method, the specimen (2 mL ascites or serum) is transferred to a sterile tube and 0.5 mL ExoQuick extracellular vesicle precipitation solution can be added and mixed. The mixture is then incubated overnight (at least 12 hours) at 4° C. and the mixture subsequently centrifuged at 10,000×g in a microfuge for 5 minutes at 4° C. The resulting precipitated extracellular vesicles can be resuspended and used to isolate IgG-bound (e.g., IgG₂-bound or protein G-recognized IgG-bound) extracellular vesicles.

### Methods of Identifying New Tumor Antigens

Also provided herein are methods for identifying new tumor antigens. These methods include identifying the proteins present within an isolated IgG-bound (e.g., IgG₂-bound or protein G-recognized IgG-bound) extracellular vesicle isolated from a subject having a cancer (e.g., ovarian cancer or any of the cancers described herein), comparing the proteins identified within the isolated IgG-bound (e.g., IgG₂-bound or protein G-recognized IgG-bound) extracellular vesicle with proteins identified in isolated IgG-unbound (e.g., IgG₂-unbound or protein G-recognized IgG-unbound) extracellular vesicles (e.g., IgG-unbound or protein G-recognized IgG-unbound extracellular vesicles isolated from the same subject or a different subject having the same type of cancer), and identifying proteins that are present in the isolated IgG-bound (e.g., IgG₂-bound or protein G-recognized IgG-bound) extracellular vesicle and not present in the isolated IgG-unbound (e.g., IgG₂-unbound or protein G-recognized IgG-unbound) extracellular vesicle as a tumor antigen.

In any of these methods, the IgG-bound (e.g., IgG₂-bound or protein G-recognized IgG-bound) extracellular vesicles are isolated using any of the methods described herein (e.g., one or more of the steps described herein). In some embodiments, the proteins present within the isolated IgG-bound (e.g., IgG₂-bound or protein G-recognized IgG-bound) extracellular vesicles are identified using one or more of the following techniques: mass spectrometry, two-dimensional gel electrophoresis, chromatography (e.g., affinity chromatography), peptide sequencing, and immunodetection. Additional methods for identifying proteins present within the isolated IgG-bound (e.g., IgG₂-bound or protein G-recognized IgG-bound) extracellular vesicles are known in the art.

In some embodiments, the isolated IgG-bound (e.g., IgG₂-bound or protein G-recognized IgG-bound) extracellular vesicles and the isolated IgG-unbound (e.g., IgG₂-unbound or protein G-recognized IgG-unbound) extracellular vesicles are isolated from a biological sample from the same subject having a cancer (e.g., a subject having ovarian cancer). In some embodiments, the isolated IgG-bound (e.g., IgG₂-bound or protein G-recognized IgG-bound) extracellular vesicles are isolated from a biological sample from a subject having a cancer (e.g., a subject having ovarian cancer), and the isolated IgG-unbound (e.g., IgG₂-unbound or protein G-recognized IgG-unbound) extracellular vesicles are isolated from a biological sample from a different subject having the same type of cancer (e.g., a subject having ovarian cancer).

Exemplary methods for isolating IgG-unbound (e.g., IgG₂-unbound or protein G-recognized IgG-unbound) extracellular vesicles are described herein. For example, in the methods described above, the flow-through or material that is not bound to an agent that specifically binds to the heavy chain constant domain of IgG (e.g., IgG₂ or protein G-recognized IgG) contains IgG-unbound (e.g., IgG₂-unbound or protein G-recognized IgG-unbound) extracellular vesicles. As described above, the proteins present within the isolated IgG-unbound (e.g., IgG₂-unbound or protein G-recognized IgG-unbound) extracellular vesicles are identified using one or more of the following techniques: mass spectrometry, two-dimensional gel electrophoresis, chromatography (e.g., affinity chromatography), peptide sequencing, and immunodetection. Additional methods for identifying proteins present within the isolated IgG-unbound (e.g., IgG₂-unbound or protein G-recognized IgG-unbound) extracellular vesicles are known in the art.

### Methods of Detecting the Presence of an IgG-Bound Extracellular Vesicle

Also provided herein are methods of detecting the presence of an IgG-bound (e.g., IgG₂-bound or protein G-recognized IgG-bound) extracellular vesicle (e.g., the presence of an IgG-bound (e.g., an IgG₂-bound or protein G-recognized IgG-bound) extracellular vesicle in a biological sample from a subject). These methods include providing a composition containing an IgG-bound (e.g., an IgG₂-bound or protein G-recognized IgG-bound) extracellular vesicle, contacting the composition with an agent that selectively binds to a heavy chain constant domain of an IgG (e.g., an agent that selectively binds to a heavy chain constant domain of IgG₂ or protein G-recognized IgG), and detecting the binding of the agent to the surface of an extracellular vesicle. In some embodiments, the agent that selectively binds to a heavy chain constant domain of an IgG is selected from Protein A, Protein G, and any of the antibodies described herein or known in the art that can bind to the heavy chain constant domain of an IgG (e.g., an antibody that selectively binds to the heavy chain constant domain of IgG₂ or protein G-recognized IgG).

In some embodiments, the composition containing an IgG-bound (e.g., an IgG₂-bound or protein G-recognized IgG-bound) extracellular vesicle is a biological sample (e.g., a biological sample obtained from any of the exemplary subjects described herein). In some embodiments, the composition is a chromatographic fraction. In some embodiments, the composition is a composition that has been partially enriched for the presence of IgG-bound (e.g., IgG₂-bound or protein G-recognized IgG-bound) extracellular vesicles. In some embodiments, the methods of detection are used to quantitate the level or concentration of IgG-bound (e.g., IgG₂-bound or protein G-recognized IgG-bound) extracellular vesicles present in a biological sample from the subject or the level or concentration of IgG-bound (e.g., IgG₂-bound or protein G-recognized IgG-bound) extracellular vesicles present in a subject.

The detecting can be performed using any of the methods described herein or known in the art. For example, the agent that selectively binds to a heavy chain constant domain of an IgG (e.g., an agent that selectively binds to a heavy chain constant domain of IgG₂ or protein G-recognized IgG) can be labeled with a fluorophore, a radioisotope, or an enzyme, and binding can be detected, for example, by measuring the light emission, light absorption, radioisotope emission, an increase in the rate or level of product formation by the enzyme, or a decrease in the level of the substrate used by the enzyme. Additional methods for detecting the binding of the agent to the IgG-bound (e.g., IgG₂-bound or protein G-recognized IgG-bound) extracellular vesicles are known in the art.

### Methods of Diagnosing a Cancer in a Subject

Also provided herein are methods of diagnosing a cancer in a subject. These methods include obtaining a sample containing a biological fluid from the subject, isolating an IgG-bound (e.g., IgG₂-bound or protein G-recognized IgG-bound) extracellular vesicle from the sample, where the IgG-bound (e.g., IgG₂-bound or protein G-recognized IgG-bound) extracellular vesicle contains an IgG (e.g., IgG₂ or protein G-recognized IgG) bound to an antigen present on the surface of the extracellular vesicle and the IgG (e.g., IgG₂ or protein G-recognized IgG) bound to the extracellular vesicle is an endogenous antibody, detecting the presence of one or more tumor antigens in the isolated IgG-bound (e.g., IgG₂-bound or protein G-recognized IgG-bound) extracellular vesicle, and diagnosing a subject having an isolated IgG-bound (e.g., IgG₂-bound or protein G-recognized IgG-bound) extracellular vesicle containing one or more (e.g., at least two, three, or four) tumor antigens as having a cancer.

In some embodiments, the subject may be suspected of having a cancer. In some embodiments, the subject may present clinically with one or more symptoms of cancer. Non-limiting examples of symptoms of cancer are described herein. Additional examples of symptoms of cancer are known in the art. In some embodiments, the subject is identified as being at risk of developing a cancer (e.g., identified as having an increased risk of cancer based on a familial history of cancer, genetic risk, and/or environmental exposure to a toxic or mutagenic substance). In some embodiments, the subject is not suspected of having cancer and does not present with one or more symptoms of a cancer. In some embodiments, the subject is not considered as being at significant risk of developing a cancer. In some embodiments, the subject is a male. In some embodiments, the subject is a female. In some embodiments, the subject may have previously had a cancer, and the subject is in remission. In some embodiments, the subject is a child (e.g., a child between the ages of 1 and 5, between the ages of 5 and 10, or between the ages of 10 and 18). In some embodiments, the subject is an adult (e.g., a person who is at least 18, 25, 30, 35, 40, 45, 50, 55, 60, 65, 70, 75, 80, 85, 90, 95, or 100 years old).

In some embodiments, the methods are performed at periodic intervals using samples obtained from the same subject (e.g., samples obtained from the same subject at least once every month, once every two months, once every four months, once every six months, once every year, or once every two years). The periodic performance of the methods described herein provides a means for monitoring a subject for the development of a cancer.

In some embodiments, the sample containing a biological fluid can contain serum, plasma, urine, cerebrospinal fluid, saliva, tears, nasal discharge, semen, amniotic fluid, vaginal discharge, lymph, tears, mucus, synovial fluid, breast milk, vitreous humor, aqueous humor, or sweat. In some embodiments, the sample comprising a biological fluid from the subject can be stored (e.g., at a temperature below 15° C., below 10° C., below 0° C., below −20° C., below −50° C., or below −70° C.) for a period of time (e.g., at least 1 hour, 12 hours, 24 hours, 36 hours, or 48 hours) prior to isolating an IgG-bound (e.g., IgG₂-bound or protein G-recognized IgG-bound) extracellular vesicle from the sample. The sample containing a biological fluid can be collected by a medical professional (e.g., a physician, a physician's assistant, a nurse, a nurse's assistant, a phlebotomist, or a laboratory worker). The sample can be obtained from a subject admitted to a health care facility (e.g., a hospital or an assisted living facility). The sample can be obtained during the periodic physical examination of a subject.

Any of the exemplary methods of isolating an IgG-bound (e.g., IgG₂-bound or protein G-recognized IgG-bound) extracellular vesicle can be performed as described herein (e.g., any combination of the various aspects described herein).

The methods encompass the diagnosis of any type of cancer. Non-limiting examples of cancer that can be diagnosed by the present methods include: bladder cancer, epithelial cancer, prostate cancer, anal cancer, appendix cancer, bone cancer, brain tumor, breast cancer, heart cancer, cervical cancer, colon cancer, gallbladder cancer, stomach cancer, head and neck cancer, liver cancer, kidney cancer, laryngeal cancer, lung cancer, ovarian cancer, pancreatic cancer, penile cancer, pituitary cancer, rectal cancer, salivary gland cancer, sarcoma, testicular cancer, throat cancer, thyroid cancer, urethral cancer, uterine cancer, vaginal cancer, or vulvar cancer.

Any method known in the art can be used to detect the presence of tumor antigens (e.g., protein or mRNA) in the isolated IgG-bound (e.g., IgG₂-bound or protein G-recognized IgG-bound) extracellular vesicles. In some embodiments, the detecting includes lysis (e.g., by sonication, detergent, heat, or pressure) of the isolated IgG-bound (e.g., IgG₂-bound or protein G-recognized IgG-bound) extracellular vesicles.

A wide variety of specific tumor antigens are known in the art. A subject can be diagnosed with a specific cancer based on the detection of the presence of one or more (e.g., two, three, four, five, six, seven, eight, nine, ten, eleven, twelve, or thirteen) tumor antigens that are associated or linked with the specific cancer in the isolated IgG-bound (e.g., IgG₂-bound or protein G-recognized IgG-bound) extracellular vesicles. For example, a subject can be diagnosed with ovarian cancer based on the detection of the presence of one or more (e.g., two, three, four, five, six, seven, eight, nine, ten, eleven, or twelve) of the tumor antigens listed in Table 1.

TABLE 1
List of Exemplary Tumor Antigens Uniquely Expressed in IgG-Bound
(e.g., IgG₂-Bound or Protein G-Recognized IgG-Bound) Extracellular
Vesicles from Subjects with Ovarian Cancer

| Name | Human Protein | Human cDNA |
|---|---|---|
| Complement 7 (C7) | SEQ ID NO: 1 | SEQ ID NO: 2 |
| Cystatin A | SEQ ID NO: 3 | SEQ ID NO: 4 |
| Alpha-1-antitrypsin | SEQ ID NO: 5 | SEQ ID NO: 6 |
| Monocyte/macrophage | SEQ ID NO: 7 | SEQ ID NO: 8 |
| Ig-related receptor-10 | | |
| (MIR-10) | | |
| Annexin A1 (ANXA1) | SEQ ID NO: 9 | SEQ ID NO: 10 |
| Annexin A2 (ANXA2) | SEQ ID NO: 11 | SEQ ID NO: 12 |
| Arginase-1 (ARG-1) | SEQ ID NO: 13 | SEQ ID NO: 14 |
| | SEQ ID NO: 15 | SEQ ID NO: 16 |
| | SEQ ID NO: 17 | SEQ ID NO: 18 |
| Complement | SEQ ID NO: 19 | SEQ ID NO: 20 |
| Component C4B-1 | | |
| Haptoglobin | SEQ ID NO: 21 | SEQ ID NO: 22 |
| Serpin peptidase | SEQ ID NO: 23 | SEQ ID NO: 24 |
| inhibitor, clade B | | |
| (Ovalbumin), member | | |
| 3 (SERPINB3) | | |
| Eukaryotic translation | SEQ ID NO: 25 | SEQ ID NO: 26 |
| elongation factor 2 | | |
| (EEF2) | | |
| Cathepsin D (CTSD) | SEQ ID NO: 27 | SEQ ID NO: 28 |

Any method known in the art can be used for detecting the presence of protein tumor antigens present in the isolated IgG-bound (e.g., IgG₂-bound or protein G-recognized IgG-bound) extracellular vesicles (e.g., using one or more antibodies that specifically bind to a specific protein tumor antigen, or a fragment thereof). For example, a sample (e.g., a sample containing a biological fluid, e.g., serum, plasma, or blood) containing IgG-bound (e.g., IgG₂-bound or protein G-recognized IgG-bound) extracellular vesicles from a subject (e.g., any of the subjects described herein) can be contacted with one or more antibodies that specifically bind to one or more of C7, cystatin-A, alpha-1-antitrypsin, MIR-10, ANXA1, ANXA2, ARG-1, complement component C4B-1, haptoglobin, SERPINB3, EEF2, CTSD, or EGFR, or an antigenic portion thereof, the binding of the one or more antibodies to proteins present in the sample can be detected using methods known in the art. In some embodiments, the detecting of the presence of protein tumor antigens in the isolated IgG-bound (e.g., IgG₂-bound or protein G-recognized IgG-bound) extracellular vesicles includes electrophoresis (e.g., two-dimensional electrophoresis) and/or mass spectrometry. In some embodiments, the detecting of the presence of protein tumor antigens in the isolated IgG-bound (e.g., IgG₂-bound or protein G-recognized IgG-bound) extracellular vesicles includes the use of an ELISA assay, a peptide array, or an aptamer that binds to a target protein or peptide.

In some embodiments, the presence of one or more mRNAs encoding a tumor antigen is detected by contacting the sample with one or more nucleic acids (e.g., primers or antisense molecules) that contain a sequence that is complementary to a contiguous sequence present in a mRNA encoding a tumor antigen (e.g., a mRNA encoding C7, cystatin-A, alpha-1-antitrypsin, MIR-10, ANXA1, ANXA2, ARG-1, complement component C4B-1, haptoglobin, SERPINB3, EEF2, CTSD, or EGFR) and, optionally, amplification is performed using a polymerase chain reaction (PCR)-based technique, as known in the art. Methods for measuring the presence or absence of a target mRNA in a biological sample are known in the art, for example, polymerase chain reaction (PCR)-based techniques (e.g., real-time quantitative PCR and gene array). Primers for use in the methods of measuring the presence or absence of a target mRNA can be designed based on the sequence of SEQ ID NO: 2, 4, 6, 8, 10, 12, 14, 16, 18, 20, 22, 24, 26, or 28, using methods known in the art.

In some embodiments all of the methods described herein, an array (e.g., any array, microarray, biochip, or point-of-care test as is known in the art) can be provided that contains one or more antibodies that specifically bind to one or more tumor antigens (e.g., one or more of C7, cystatin-A, alpha-1-antitrypsin, MIR-10, ANXA1, ANXA2, ARG-1, complement component C4B-1, haptoglobin, SERPINB3, EEF2, CTSD, and EGFR), and the array can be contacted with the contents of the isolated IgG-bound (e.g., IgG₂-bound or protein G-recognized IgG-bound) extracellular vesicles, and the binding of any proteins present in the sample can be detected. Likewise, an array can be provided that contains one or more nucleic acids (e.g., probes) that contain a sequence complementary to a contiguous sequence present in a mRNA encoding a tumor antigen (e.g., C7, cystatin-A, alpha-1-antitrypsin, MIR-10, ANXA1, ANXA2, ARG-1, complement component C4B-1, haptoglobin, SERPINB3, EEF2, CTSD, or EGFR). The arrays can be used to develop a database of information using data obtained using the methods described herein.

Methods for detecting binding of the antibodies to tumor antigens are known in the art, and can include the use of secondary antibodies. The secondary antibodies are generally modified to be detectable, e.g., labeled. The term “labeled” is intended to encompass direct labeling by coupling (i.e., physically linking) a detectable substance to the secondary antibody, as well as indirect labeling of the multimeric antigen by reactivity with a detectable substance. Examples of detectable substances include various enzymes, prosthetic groups, fluorescent materials, luminescent materials, bioluminescent materials, and radioactive materials. Examples of suitable enzymes include horseradish peroxidase (HRP), alkaline phosphatase, β-galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, and quantum dots, dichlorotriazinylamine fluorescein, dansyl chloride, and phycoerythrin; an example of a luminescent material includes luminol; examples of bioluminescent materials include green fluorescent protein and variants thereof, luciferase, luciferin, and aequorin; and examples of suitable radioactive material include ¹²⁵I, ¹³¹I, ³⁵S, or ³H. Methods for producing such labeled antibodies are known in the art, and many are commercially available.

Any method of detecting protein tumor antigens present in the isolated IgG-bound (e.g., IgG₂-bound or protein G-recognized IgG-bound) extracellular vesicles can be used, including but not limited to radioimmunoassays (RIA), enzyme-linked immunosorbent assays (ELISA), Western blotting, surface plasmon resonance, microfluidic devices, protein array, protein purification (e.g., chromatography, such as affinity chromatography), mass spectrometry, two-dimensional gel electrophoresis, or other assays as known in the art. In some embodiments, the detecting of the presence of protein tumor antigens in the isolated IgG-bound (e.g., IgG₂-bound or protein G-recognized IgG-bound) extracellular vesicles includes the use of an ELISA assay, a peptide array, or an aptamer that binds to a target protein or peptide.

The term “array,” as used herein, generally refers to a predetermined spatial arrangement of binding ligands (e.g., antibodies or nucleic acid probes) or spatial arrangements of binding ligands or antigens. Arrays according to the present invention include antibodies or nucleic acid probes immobilized on a surface may also be referred to as “antibody arrays” or “gene arrays,” respectively. Arrays according to the present invention that comprise surfaces activated, adapted, prepared, or modified to facilitate the binding of sample proteins or mRNAs to the surface may also be referred to as “binding arrays.” Further, the term “array” can be used herein to refer to multiple arrays arranged on a surface, such as would be the case where a surface bore multiple copies of an array. Such surfaces bearing multiple arrays may also be referred to as “multiple arrays” or “repeating arrays.” The use of the term “array” herein can encompass antibody arrays, gene arrays, binding arrays, multiple arrays, and any combination thereof; the appropriate meaning will be apparent from context. An array can include antibodies that detect protein tumor antigens or nucleic acid probes that detect mRNAs encoding a tumor antigen. The array can be contacted with an isolated IgG-bound (e.g., IgG₂-bound or protein G-recognized IgG-bound) extracellular vesicle, the lysate of an isolated IgG-bound (e.g., IgG₂-bound or protein G-recognized IgG-bound) extracellular vesicle, or the proteins or mRNAs isolated from the IgG-bound (e.g., IgG₂-bound or protein G-recognized IgG-bound) extracellular vesicle.

An array of the invention contains a substrate. By “substrate” or “solid support” or other grammatical equivalents, herein is meant any material appropriate for the attachment of antibodies or nucleic acid probes and is amenable to at least one detection method. As will be appreciated by those in the art, the number of possible substrates is very large. Possible substrates include, but are not limited to, glass and modified or functionalized glass, plastics (including acrylics, polystyrene, and copolymers of styrene and other materials, polypropylene, polyethylene, polybutylene, polyurethanes, TEFLON®, etc.), polysaccharides, nylon or nitrocellulose, resins, silica or silica-based materials including silicon and modified silicon, carbon, metals, inorganic glasses, plastics, ceramics, and a variety of other polymers. In addition, as is known the art, the substrate can be coated with any number of materials, including polymers, such as dextrans, acrylamides, gelatins, or agarose. Such coatings can facilitate the use of the array with a sample derived from a biological fluid, e.g., urine, plasma, or serum.

A planar array of the invention will generally contain addressable locations (e.g., “pads,” “addresses,” or “micro-locations”) of antibodies or nucleic acid probes in an array format. The size of the array will depend on the composition and end use of the array. The arrays can contain one, two, or more different antibodies or nucleic acid probes. Generally, the array will comprise from two to as many as 20 different antibodies or nucleic acid probes, depending on the end use of the array. A microarray of the invention will generally contain at least one antibody or nucleic acid probe that identifies or “captures” a target protein or mRNA present in a biological sample. In some embodiments, the compositions of the invention may not be in an array format; that is, for some embodiments, compositions comprising a single antibody or nucleic acid probe can be made as well. In addition, in some arrays, multiple substrates can be used, either of different or identical compositions. Thus, for example, large planar arrays can comprise a plurality of smaller substrates.

As an alternative to planar arrays, bead-based assays in combination with flow cytometry have been developed to perform multiparametric immunoassays. In bead-based assay systems, one or more antibodies can be immobilized on addressable microspheres. Each antibody for each individual immunoassay is coupled to a distinct type of microsphere (i.e., “microbead”) and the immunoassay reaction takes place on the surface of the microspheres. Dye-linked microspheres with discrete fluorescence intensities are loaded separately with their appropriate biomolecules. The different bead sets carrying different capture probes (e.g., antibodies) can be pooled as necessary to generate custom bead arrays. Bead arrays are then incubated with the sample in a single reaction vessel to perform the immunoassay.

In some embodiments, product formation of the tumor antigen with an antibody can be detected with a fluorescence-based reporter system. The antibodies can be labeled directly by a fluorogen or detected by a second fluorescently-labeled capture biomolecule. The signal intensities derived from target-bound antibodies are measured in a flow cytometer. The flow cytometer first identifies each microsphere by its individual color code. Second the amount of antibody on each individual bead is measured by the second color fluorescence specific for the bound target. This allows multiplexed quantitation of multiple targets from a single sample within the same experiment. Sensitivity, reliability, and accuracy are comparable to standard microtiter ELISA procedures. With bead-based immunoassay systems, proteins can be simultaneously quantified from biological samples. An advantage of bead-based systems is the individual coupling of an antibody to distinct microspheres.

Thus, microbead array technology can be used to sort proteins bound to specific antibodies using a plurality of microbeads, each of which can carry about 100,000 identical molecules of a specific antibody on its surface. Once captured, the protein can be handled as a fluid, referred to herein as a “fluid microarray.”

An array can encompass any means for detecting a protein. For example, microarrays can be biochips that provide high-density immobilized arrays of antibodies, where antibody binding is monitored indirectly (e.g., via fluorescence). In addition, an array can be of a format that involves the capture of target proteins by biochemical or intermolecular interaction, coupled with direct detection by mass spectrometry (MS).

Arrays and microarrays that can be used with the methods described herein can be made according to the methods described in U.S. Pat. Nos. 6,329,209; 6,365,418; 6,406,921; 6,475,808; and 6,475,809, which are incorporated herein in their entirety. New arrays, to detect specific selections or sets of tumor antigens described herein can also be made using the methods described in these patents.

The antibodies can be immobilized on the surface using methods and materials that minimize the denaturing of the antibodies, that minimize alterations in the structure of the antibodies, or that minimize interactions between the antibodies and the surface on which they are immobilized.

Surfaces useful in the arrays can be of any desired shape (form) and size. Non-limiting examples of surfaces include chips, continuous surfaces, curved surfaces, flexible surfaces, films, plates, sheets, tubes, and the like. Surfaces preferably have areas ranging from approximately a square micron to approximately 500 cm². The area, length, and width of surfaces according to the present invention can be varied according to the requirements of the assay to be performed. Considerations may include, for example, ease of handling, limitations of the material(s) of which the surface is formed, requirements of detection systems, requirements of deposition systems (e.g., arrayers), and the like.

In certain embodiments, it is desirable to employ a physical means for separating groups or arrays of binding islands or immobilized antibodies or nucleic acid probes: such physical separation facilitates exposure of different groups or arrays to different solutions of interest. Therefore, in certain embodiments, arrays are situated within wells of 96-, 384-, 1536-, or 3456-microwell plates. In such embodiments, the bottoms of the wells can serve as surfaces for the formation of arrays, or arrays can be formed on other surfaces and then placed into wells. In certain embodiments, such as where a surface without wells is used, binding islands can be formed or antibodies or nucleic acid probes can be immobilized on a surface and a gasket having holes spatially arranged so that they correspond to the islands or antibodies/nucleic acid probes can be placed on the surface. Such a gasket is preferably liquid-tight. A gasket can be placed on a surface at any time during the process of making the array and can be removed if separation of groups or arrays is no longer necessary.

Modifications or binding of target proteins or mRNAs to antibodies or nucleic acid probes in solution or immobilized on an array can be detected using detection techniques known in the art. Examples of such techniques include immunological techniques such as competitive binding assays and sandwich assays; fluorescence detection using instruments such as confocal scanners, confocal microscopes, or CCD-based systems, and techniques such as fluorescence, fluorescence polarization (FP), fluorescence resonant energy transfer (FRET), total internal reflection fluorescence (TIRF), fluorescence correlation spectroscopy (FCS); colorimetric/spectrometric techniques; surface plasmon resonance, by which changes in mass of materials adsorbed at surfaces can be measured; techniques using radioisotopes, including conventional radioisotope binding and scintillation proximity assays (SPA); mass spectroscopy, such as matrix-assisted laser desorption/ionization mass spectroscopy (MALDI) and MALDI-time of flight (TOF) mass spectroscopy; ellipsometry, which is an optical method of measuring thickness of protein films; quartz crystal microbalance (QCM), a very sensitive method for measuring mass of materials adsorbing to surfaces; scanning probe microscopies, such as atomic force microscopy (AFM) and scanning electron microscopy (SEM); and techniques such as electrochemical, impedance, acoustic, microwave, and infrared (IR)/Raman detection. See, e.g., Mere et al., “Miniaturized FRET assays and microfluidics: key components for ultra-high-throughput screening,” Drug Discovery Today 4(8):363-369, 1999, and references cited therein; Lakowicz, J. R., Principles of Fluorescence Spectroscopy, 2nd Edition, Plenum Press, 1999.

Arrays as described herein can be included in kits. Such kits can also include, as non-limiting examples, one or more of: reagents useful for preparing antibodies or nucleic acid probes for immobilization onto binding islands or areas of an array, reagents useful in preparing a sample, reagents useful for isolating IgG-bound (e.g., IgG₂-bound or protein G-recognized IgG-bound) extracellular vesicles, reagents useful for lysing or disrupting IgG-bound (e.g., IgG₂-bound or protein G-recognized IgG-bound) extracellular vesicles, reagents useful for detecting binding of target proteins or mRNAs in an IgG-bound (e.g., IgG₂-bound or protein G-recognized IgG-bound) extracellular vesicle to immobilized antibodies or nucleic acid probes, control samples that include purified target proteins or mRNAs, and/or instructions for use.

For example, kits useful in the methods described herein can include one or more (e.g., two, three, four, five, six, seven, eight, nine, ten, eleven, twelve, or thirteen) antibodies or nucleic acid probes (e.g., a sequence complementary to a contiguous sequence present in a target mRNA) that specifically bind to C7, cystatin-A, alpha-1-antitrypsin, MIR-10, ANXA1, ANXA2, ARG-1, complement component C4B-1, haptoglobin, SERPINB3, EEF2, CTSD, or EGFR (a protein or protein fragment, or an mRNA). For example, the one or more antibodies or the one or more nucleic acid probes provided in the kits can be immobilized on a surface (e.g., in the form of an ELISA assay or a gene-chip array).

Total mRNA can be purified from the isolated IgG-bound (e.g., IgG₂-bound or protein G-recognized IgG-bound) extracellular vesicles using methods known in the art, for example, TRIZOL (Invitrogen, Inc.) according to manufacturer's instructions (Invitrogen, Inc.), except with the isopropanol precipitation step extended to overnight. The mRNA quality and yield can be accessed using a GENEQUANT II. Methods for analyzing purified mRNAs are known in the art and include reverse transcription PCR, gene array analysis, and Northern blotting.

Proteins can be purified from the isolated IgG-bound (e.g., IgG₂-bound or protein G-recognized IgG-bound) extracellular vesicles, for example, by continuing the TRIZOL (Invitrogen, Inc.) isolation procedure, as described by the manufacturer. In some embodiments, the quantity of protein can be determined by the Bradford microassay method, using BSA as a standard. Any protein or mRNA isolation methods described herein or known in the art can be used to detect the presence or absence of a protein tumor antigen or mRNA encoding a tumor antigen in the isolated IgG-bound (e.g., IgG₂-bound or protein G-recognized IgG-bound) extracellular vesicles.

In any of the methods described herein, the detection of the presence (e.g., a level above a threshold, e.g., detectable, level) of one or more (e.g., one, two, three, four, five, six, seven, eight, nine, ten, eleven, twelve, or thirteen) of C7, cystatin-A, alpha-1-antitrypsin, MIR-10, ANXA1, ANXA2, ARG-1, complement component C4B-1, haptoglobin, SERPINB3, EEF2, CTSD, and EGFR (protein or mRNA) in an isolated IgG-bound (e.g., IgG₂-bound or protein G-recognized IgG-bound) extracellular vesicle, indicates that the subject has ovarian cancer (e.g., ovarian cancer).

Some embodiments further include updating a subject's clinical records to indicate the diagnosis. Some embodiments further include performing one or more additional clinical blood tests for a cancer (e.g., prostate-specific antigen). Some embodiments further include administering a cancer therapeutic (e.g., any of the cancer therapeutics described herein or known in the art) to a subject identified as having an isolated IgG-bound (e.g., IgG₂-bound or protein G-recognized IgG-bound) extracellular vesicle containing one or more tumor antigens (e.g., a subject that is not diagnosed as having cancer and/or does not present clinically with one or more symptoms of cancer). Some embodiments further include performing exploratory surgery to resect the cancer from the subject. Some embodiments further include modifying a computer database to indicate the subject's diagnosis with a cancer.

### Methods of Treating a Subject or Selecting a Treatment for a Subject

Methods of treating a subject are also provided. In some embodiments, these methods include selectively administering one or more (e.g., two, three, or four) cancer therapeutics to a subject that has been determined to have an IgG-bound (e.g., IgG₂-bound or protein G-recognized IgG-bound) extracellular vesicle that contains one or more (e.g., at least two, three, four, five, six, seven, eight, nine, ten, eleven, twelve, or thirteen) tumor antigens, where the IgG-bound (e.g., IgG₂-bound or protein G-recognized IgG-bound) extracellular vesicle contains an IgG (e.g., IgG₂ or protein G-recognized IgG) bound to an antigen present on the surface of the extracellular vesicle and the IgG (e.g., IgG₂ or protein G-recognized IgG) bound to the extracellular vesicle is an endogenous antibody. In some embodiments, these methods include obtaining a sample comprising a biological fluid from the subject, isolating an IgG-bound (e.g., IgG₂-bound or protein G-recognized IgG-bound) extracellular vesicle from the sample, where the IgG-bound (e.g., IgG₂-bound or protein G-recognized IgG-bound) extracellular vesicle contains an IgG (e.g., IgG₂ or protein G-recognized IgG) bound to an antigen present on the surface of the extracellular vesicle and the IgG (e.g., IgG₂ or protein G-recognized IgG) bound to the extracellular vesicle is an endogenous antibody, detecting the presence of one or more tumor antigens in the isolated IgG-bound (e.g., IgG₂-bound or protein G-recognized IgG-bound) extracellular vesicle, selecting a subject having an IgG-bound (e.g., IgG₂-bound or protein G-recognized IgG-bound) extracellular vesicle containing one or more tumor antigens, and administering one or more cancer therapeutics to the selected subject.

Any of the exemplary samples, subjects, methods of isolating an IgG-bound (e.g., IgG₂-bound or protein G-recognized IgG-bound) extracellular vesicle, methods of detecting the presence of one or more tumor antigen(s) in an IgG-bound (e.g., IgG₂-bound or protein G-recognized IgG-bound) extracellular vesicle, and tumor antigens described herein can be used in these methods in any combination without limitation. In some embodiments, the subject may not present clinically with one or more symptoms of a cancer or may be identified as having a low risk of developing cancer (e.g., no familial history of cancer, no known exposure to toxins and/or mutagens, and/or no other known genetic mutations that are associated with a cancer). In some embodiments, the subject presents clinically with one or more symptoms of a cancer or has a high risk of developing cancer (e.g., familial history of cancer, known exposure to toxins and/or mutagens, and/or known genetic mutations that are associated with a cancer).

Examples of cancer therapeutics include, without limitation, an antimetabolite, an alkylating agent, interleukin-2, a therapeutic antibody, radiation, or hormone deprivation therapy (e.g., androgen deprivation therapy and estrogen blockers (e.g., tamoxifen, toremifene, fluvestrant, letrozole, anastrozole, exemestane, goserelin, leuprolide, and megestrol acetate). Non-limiting examples of antimetabolites include methotrexate, trimetrexate, pentostatin, cytarabine, fludarabine phosphate, hydroxyurea, fluorouracil, floxuridine, chlorodeoxyadenosine, gemcitabine, thioguanine, and 6-mercaptopurine. Non-limiting examples of alkylating agents include lomustine, carmustine, streptozocin, mechlorethamine, melphalan, uracil nitrogen mustard, chlorambucil, cyclophosphamide, iphosphamide, cisplatin, carboplatin, mitomycin, thiotepa, dacarbazin, procarbazine, hexamethyl melamine, triethylene melamine, busulfan, pipobroman, and mitotane. Non-limiting examples of therapeutic antibodies include ipilimumab and trastuzumab. Additional exemplary cancer therapeutics include bleomycin, topotecan, irinotecan, camptothecin, daunorubicin, doxorubicin, idarubicin, mitoxantrone, teniposide, etoposide, dactinomycin, mithramycin, vinblastine, vincristine, navelbine, paclitaxel, and docetaxel. In some embodiments, a subject is identified as having ovarian cancer (e.g., using the diagnostic methods described herein) and administered a cancer therapeutic selected from the group of doxorubicin and topotecan. One or more (e.g., two, three, four, or five) cancer therapeutics can be administered to the subject.

The therapeutic treatment can be administered by a health care professional (e.g., a physician, a nurse, or a physician's assistant). The treatment can be administered in a patient's home or in a heath care facility (e.g., a hospital or a clinic). The one or more cancer therapeutics can be administered orally, subcutaneously, intramuscularly, intravenously, intraarterially, intrathecally, or intraperitoneally.

The dosage and selection of the cancer therapeutic can be determined by a health care professional based on known in the art. See, e.g., Abraham et al., The Bethesda Handbook of Clinical Oncology (Lippincott Williams & Wilkins; Third edition, Sep. 4, 2009); Casciato and Territo, Manual of Clinical Oncology (Lippincott Manual Series) (Lippincott Williams & Wilkins; Sixth, North American Edition, Sep. 5, 2008); Haffty and Wilson, Handbook of Radiation Oncology: Basic Principles and Clinical Protocols, (Jones & Bartlett Publishers; 1st Edition, Jul. 23, 2008); and Abeloff et al., Abeloffs Clinical Oncology: Expert Consult (Churchill Livingstone; 4th Edition, May 21, 2008); Feig et al., The M.D. Anderson Surgical Oncology Handbook (Lippincott Williams & Wilkins; 4th Edition (Jun. 21, 2006). For example, a single dose of a cancer therapeutic can contain between 1 mg to 500 mg of the therapeutic agent (e.g., between 10 mg and 400 mg, between 10 mg and 300 mg, between 1 mg and 200 mg, between 1 mg and 100 mg, between 1 mg and 50 mg, or between 1 mg and 25 mg).

The one or more cancer therapeutic can be administered to the subject with a frequency of at least once a day, at least twice a day, at least once a week, at least once every two weeks, at least once every month, or at least once every two months. In some embodiments, the one or more cancer therapeutics can be administered to the subject for a treatment period of at least one day (e.g., at least two days, at least three days, at least four days, at least five days, at least six days, at least one week, at least two weeks, or at least one month).

### Methods for Selecting a Subject for a Clinical Trial

Also provided are methods of selecting a subject for participation in a clinical trial that include obtaining a sample containing a biological fluid from a subject, isolating an IgG-bound (e.g., IgG₂-bound or protein G-recognized IgG-bound) extacellular vesicle from the sample, where the IgG-bound (e.g., IgG₂-bound or protein G-recognized IgG-bound) extracellular vesicle contains an IgG (e.g., IgG₂ or protein G-recognized IgG) bound to an antigen present on the surface of the extracellular vesicle and the IgG (e.g., IgG₂ or protein G-recognized IgG) bound to the extracellular vesicle is an endogenous antibody, detecting the presence of one or more tumor antigens in the isolated IgG-bound (e.g., IgG₂-bound or protein G-recognized IgG-bound) extracellular vesicles, and selecting a subject having an IgG-bound (e.g., IgG₂-bound or protein G-recognized IgG-bound) extracellular vesicle containing one or more tumor antigens for participation in a clinical study.

Any of the exemplary samples, subjects, methods of isolating an IgG-bound (e.g., IgG₂-bound or protein G-recognized IgG-bound) extracellular vesicle, methods of detecting the presence of one or more tumor antigen(s) in an IgG-bound (e.g., IgG₂-bound or protein G-recognized IgG-bound) extacellular vesicle, and tumor antigens described herein can be used in these methods in any combination without limitation. Exemplary clinical studies include administering a cancer therapeutic to a subject (e.g., the administering of one or more ovarian cancer therapeutics to a subject).

### EXAMPLES

The invention is further described in the following examples, which do not limit the scope of the invention described in the claims.

### Example 1. Isolation of Extracellular Vesicle Reactive IgG

Tumor-derived extacellular vesicles are released into the blood of cancer patients, and are present, for example, at ˜2-5×10¹⁰/mL in the peripheral circulation of ovarian cancer patients. The exosomal protein profiles from tumor-derived plasma extracellular vesicles contain approximate representations of the proteome of the original tumor cell. Isolated extracellular vesicle-reactive IgG (e.g., IgG₂ or protein G-recognized IgG) can be used to identify new tumor antigens.

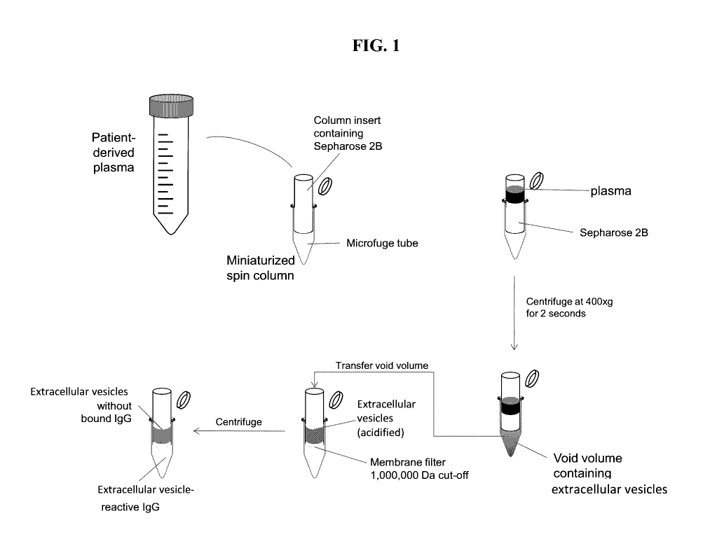

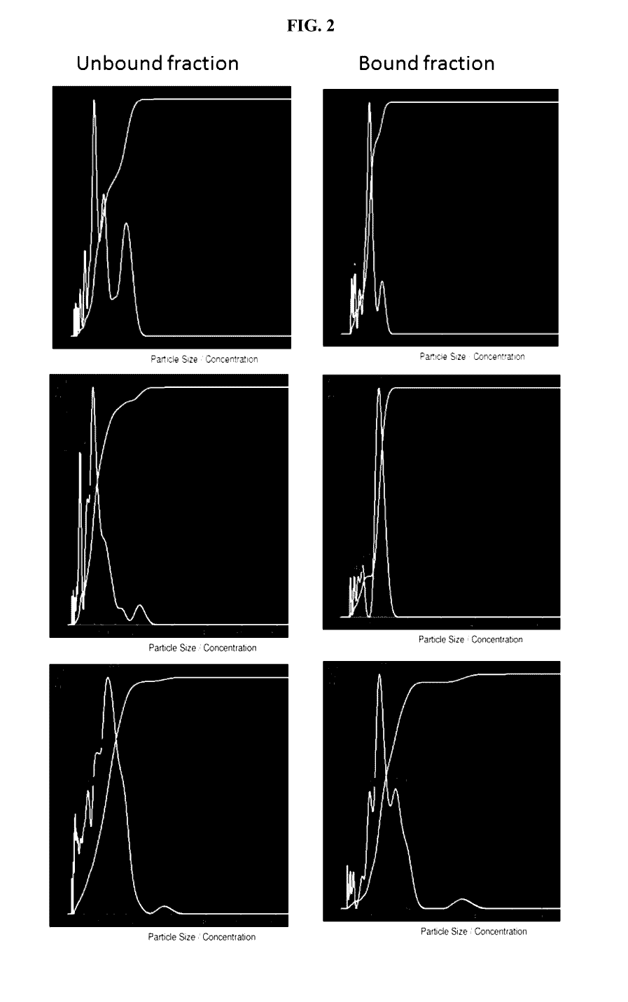

Extracellular vesicle-reactive IgG (e.g., IgG₂ or protein G-recognized IgG) was isolated from plasma obtained from ovarian cancer patients using the following procedure (FIG. 1). A miniature Sepharose 2B column was generated by fitting a 30 μm-filter into a microfuge spin column, and then adding 0.6 mL of Sepharose 2B on top of the insert. The column was washed with Tris-buffered saline (TBS) and centrifuged for 2 seconds at 800 rpm, before plasma (0.25 mL) from an ovarian cancer patient was applied to the top of the column. The column was centrifuged for 2 seconds after the plasma was applied to the column. TBS (0.1 mL) was added to the top of the column and centrifuged again for 2 seconds at 800 rpm. The flow-through (representing the void volume) was removed from the lower chamber (below the filter). An additional 0.1 mL TBS was added to the top of the column and recentrifuged for 2 seconds. The additional resulting flow-through was removed from the lower chamber and pooled with the first sample. Western immunoblotting of the resulting pooled sample demonstrated the presence of the extracellular vesicle marker CD63.

The resulting pooled material was added to the insert of a second spin column. This second spin column contained a 1,000 kDa cut-off membrane. The pooled material was acidified by the addition of 10× glycine-HCl, pH 2.5 (0.1 M final concentration) before it was applied to the second spin column. The acidified pooled material was incubated for 20 minutes at 4° C., then placed on the top of the second column, and centrifuged. After centrifugation, the upper chamber contained IgG-unbound (e.g., IgG₂-unbound or protein G-recognized IgG-unbound) extacellular vesicles and the lower chamber contained the extracellular vesicle-reactive IgG (e.g., IgG₂ or protein G-recognized IgG). The obtained extracellular vesicle-reactive IgG (e.g., IgG₂ or protein G-recognized IgG) can be used to determine the identity of tumor antigens present in the extracellular vesicles from the ovarian cancer patient. Extacellular vesicle-reactive IgG (e.g., IgG₂ or protein G-recognized IgG) can be isolated from different subjects having different types of cancer, and can likewise be used to identify new tumor antigens in a variety of different types of cancer.

### Example 2. Isolation and Characterization IgG-Bound Extacellular Vesicles

An experiment was performed to isolate IgG-bound (e.g., IgG₂-bound or protein G-recognized IgG-bound) extacellular vesicles from sera obtained from patients having ovarian cancer. The IgG-bound (e.g., IgG₂-bound or protein G-recognized IgG-bound) extacellular vesicles were isolated using the following chromatography procedure. Sera from patients having ovarian cancer (1 mL) was separated on Sepharose 2B microfuge spin columns as described in Example 1. The elution from the Sepharose 2B microfuge spin columns was monitored at 280 nm. The vesicular material obtained was examined using a Nanosight LM10 instrument to confirm the size distribution of the extacellular vesicles.

IgG-bound (e.g., IgG₂-bound or protein G-recognized IgG-bound) extacellular vesicles were isolated from the resulting extacellular vesicle pool using a microspin column containing Protein G beads. The volume of extracellular vesicle pool applied to the microspin column containing Protein G beads was 500 μL (due to the volume of the tube), and the column was washed three times with 1 mL of PBS. The IgG-bound (e.g., IgG₂-bound or protein G-recognized IgG-bound) extracellular vesicles were eluted from the Protein G beads using three 0.5-mL aliquots of 0.1 M glycine-HCl, neutralized with Tris base.

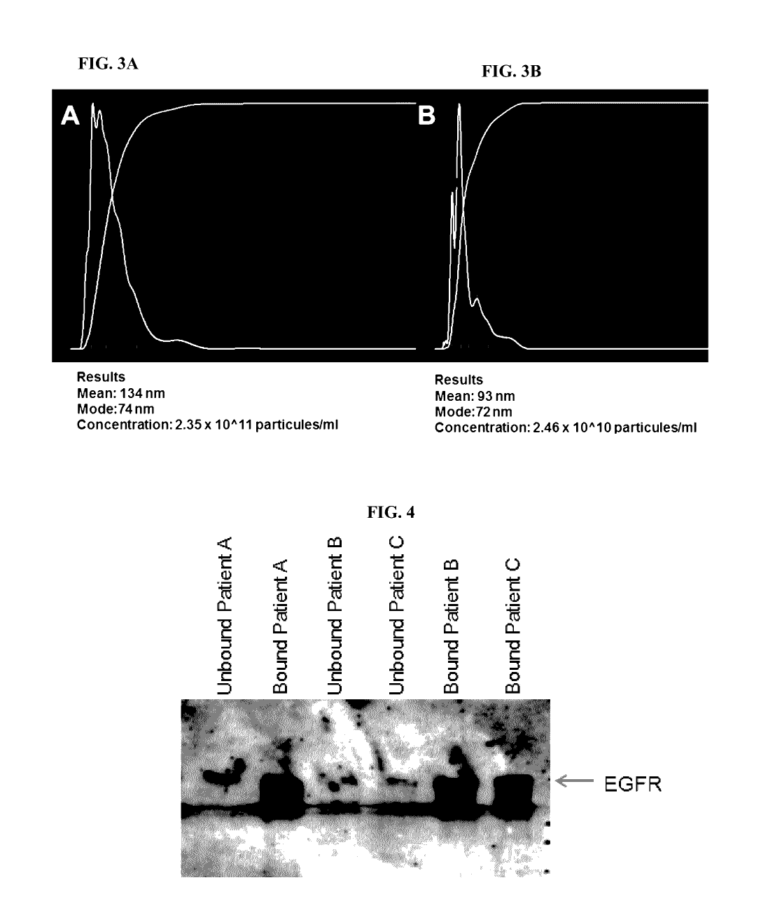

A Nanosight LM10 instrument was used to confirm the size distribution of the resulting IgG-unbound (e.g., IgG₂-unbound or protein G-recognized IgG-unbound) extacellular vesicle and the IgG-bound (e.g., IgG₂-bound or protein G-recognized IgG-bound) extacellular vesicle populations (FIG. 2, right and left panels respectively). The isolated IgG-bound (e.g., IgG₂-bound or protein G-recognized IgG-bound) extracellular vesicles represent a subpopulation of circulating extacellular vesicles having a mean diameter of 72 nm (compare FIG. 3A and FIG. 3B). The number of IgG-bound (e.g., IgG₂-bound or protein G-recognized IgG-bound) extacellular vesicles was determined to be 2.46×10¹⁰ vesicles/mL using Nanosight analysis.

The isolated IgG-bound (e.g., IgG₂-bound or protein G-recognized IgG-bound) extacellular vesicles were shown to contain an elevated level of a tumor antigens (EGFR protein) compared to IgG-unbound (e.g., IgG₂-unbound or protein G-recognized IgG-unbound) extacellular vesicles (FIG. 4). Subpopulations of IgG-bound (e.g., IgG₂-bound or protein G-recognized IgG-bound) extacellular vesicles and IgG-unbound (e.g., IgG₂-unbound or protein G-recognized IgG-unbound) extacellular vesicles were isolated from the serum of two different ovarian cancer subjects (hereafter referred to as the “First” and “Second” Ovarian Cancer Subject).

The extacellular vesicles in each population were pelleted by precipitation with ExoQuick (SBI, Mountain View, Calif.). ExoQuick was added to each extracellular vesicle-containing population at a 1:5 (v/v) dilution, incubated overnight, and pelleted by centrifugation at 10,000×g for 5 minutes. Pelleting of each extacellular vesicle population was assessed by assaying protein concentrations in both the pellet and supernatant. The resulting pellet was then subjected to extraction with TRIZOL (Invitrogen, Inc.) by the manufacturer's instructions, except that the isopropanol precipitation of RNA was extended to overnight at 4° C. The extraction procedure was continued for protein isolation.

Exosomal protein fractions from the TRIZOL (Invitrogen, Inc.) extraction were initially separated by SDS-PAGE or gel electrophoresis. Protein spots were excised and processed for protein identification by tandem mass spectrometry (MS). The exosomal proteins in each population were also analyzed using linear ion trap mass spectrometry (LTQ, Thermo Electron Corp) at the University of Louisville's Core Proteomics Laboratory. The protein in each spot was digested using trypsin, eluted from the reverse phase (RP)-HPLC, and analyzed by light triggered and quenched (LTQ) measurement. All MS/MS samples were evaluated using Sequest (ThermoFinnigan, San Jose, Calif.). Sequest was set-up assuming trypsin digestion. The profiles for each sample were obtained and analyzed with Scaffold 2_06_02 (Proteosome Software Inc., Portland, Oreg.), which validated MS/MS-based protein identification. Protein identifications were accepted if they could be established at greater than 99.0% probability and contained at least 2 identified peptides. Only proteins identified with 2 or more peptides and an expected value of less than 0.05 were included, since these criteria produce a false discovery rate (FDR) of 0%. Protein probabilities were assigned by the Protein Prophet's algorithm for protein prediction.

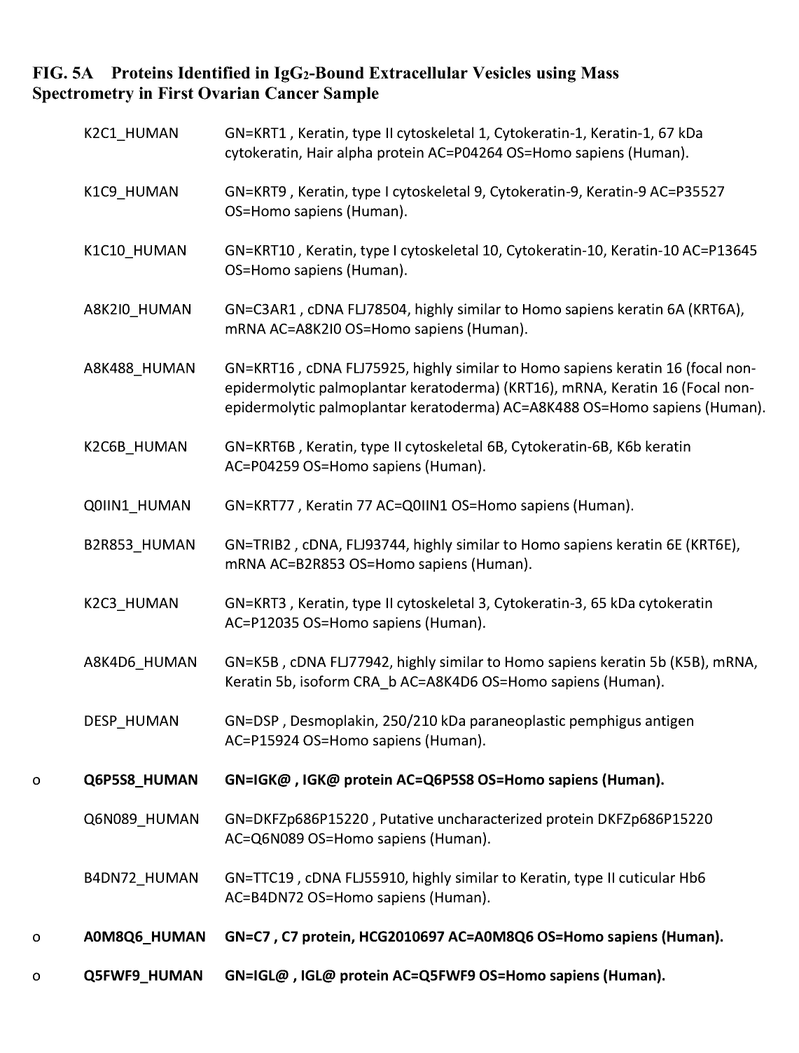

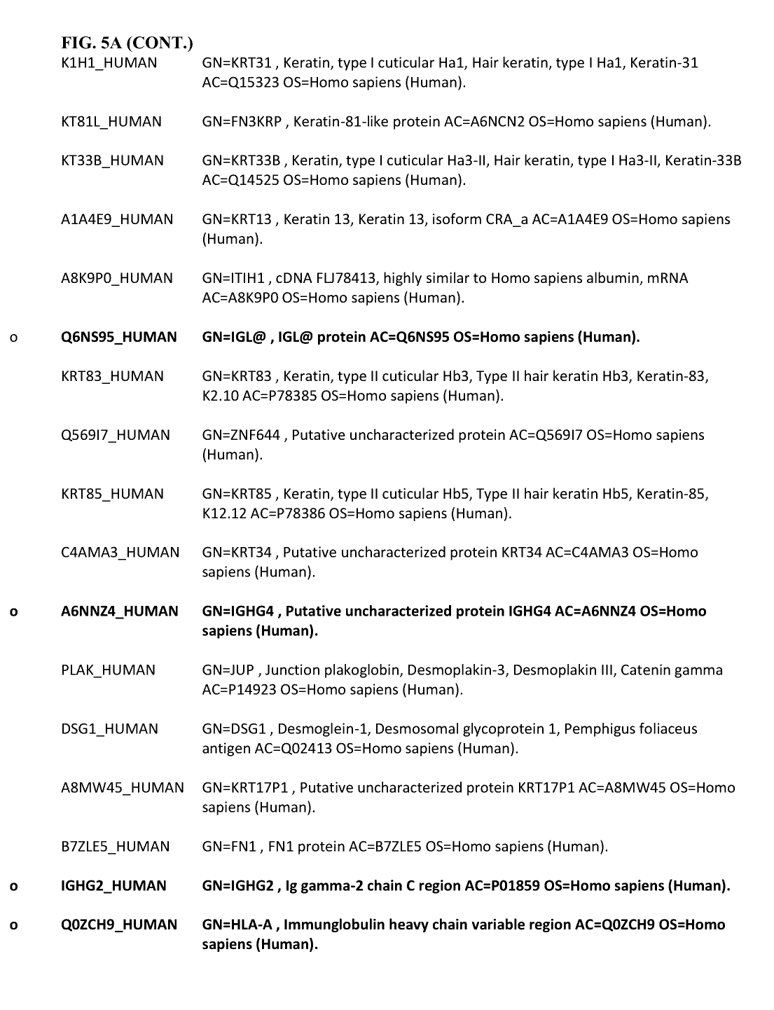

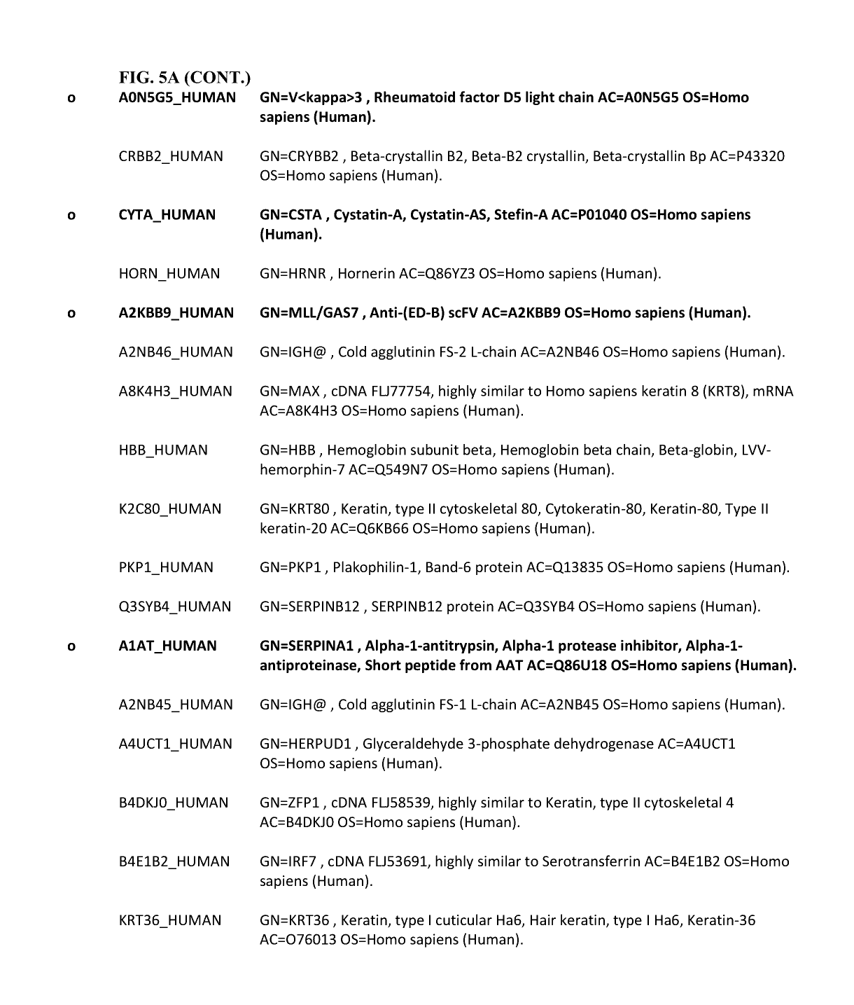

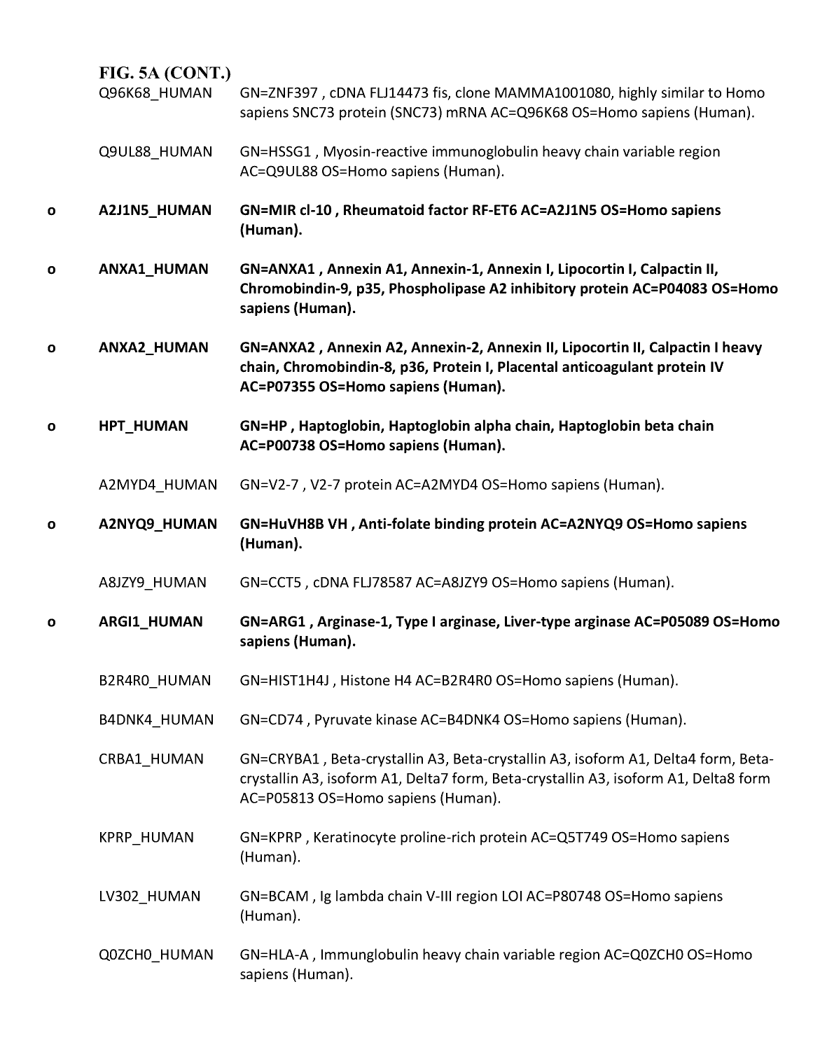

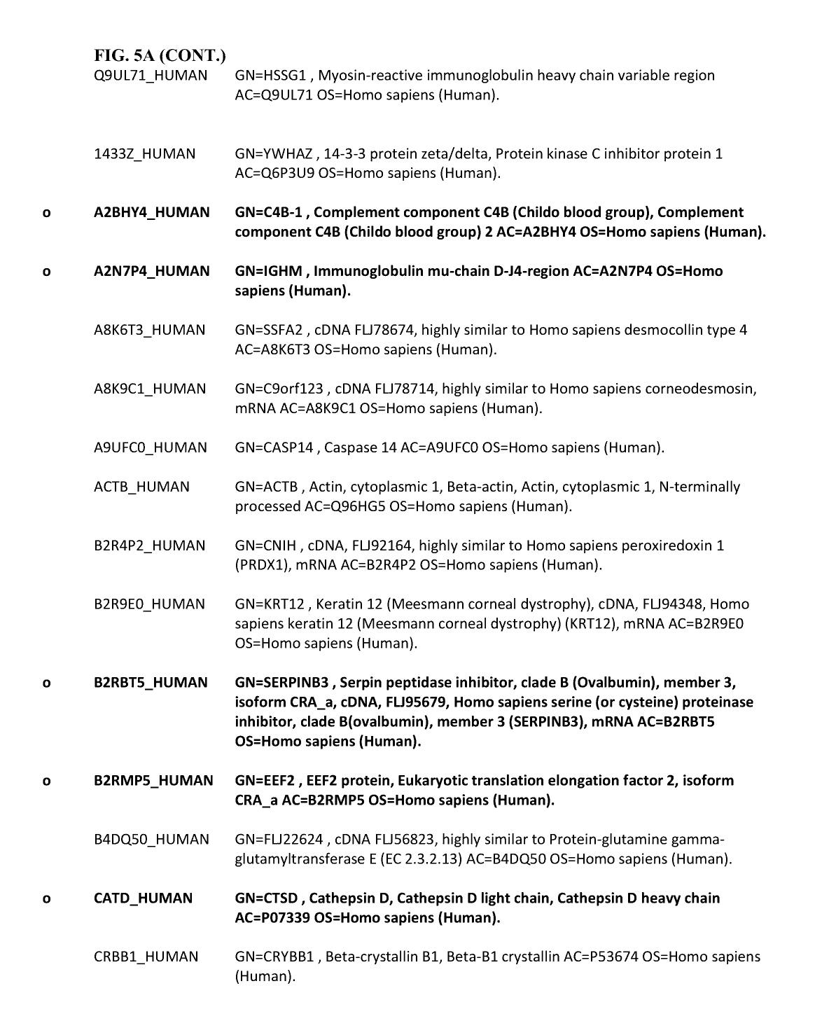

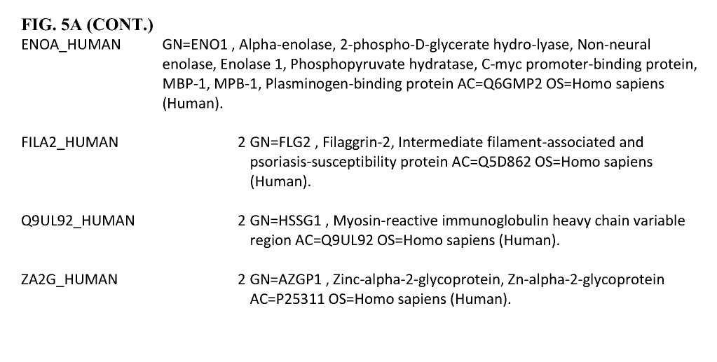

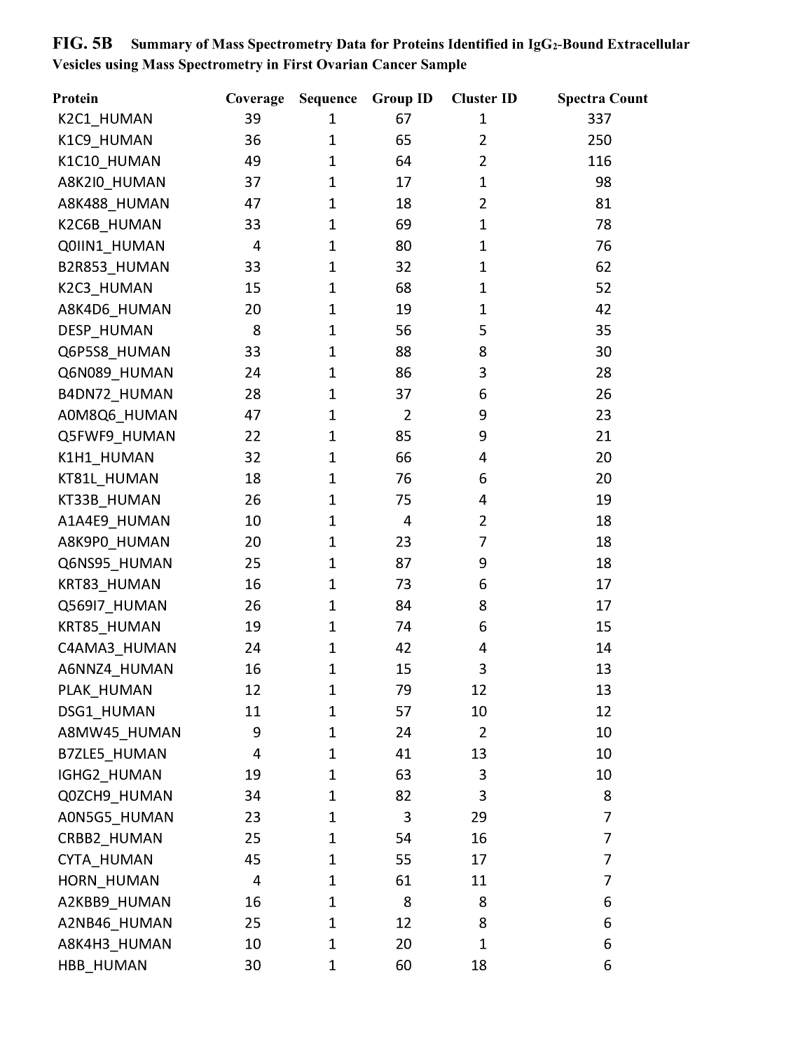

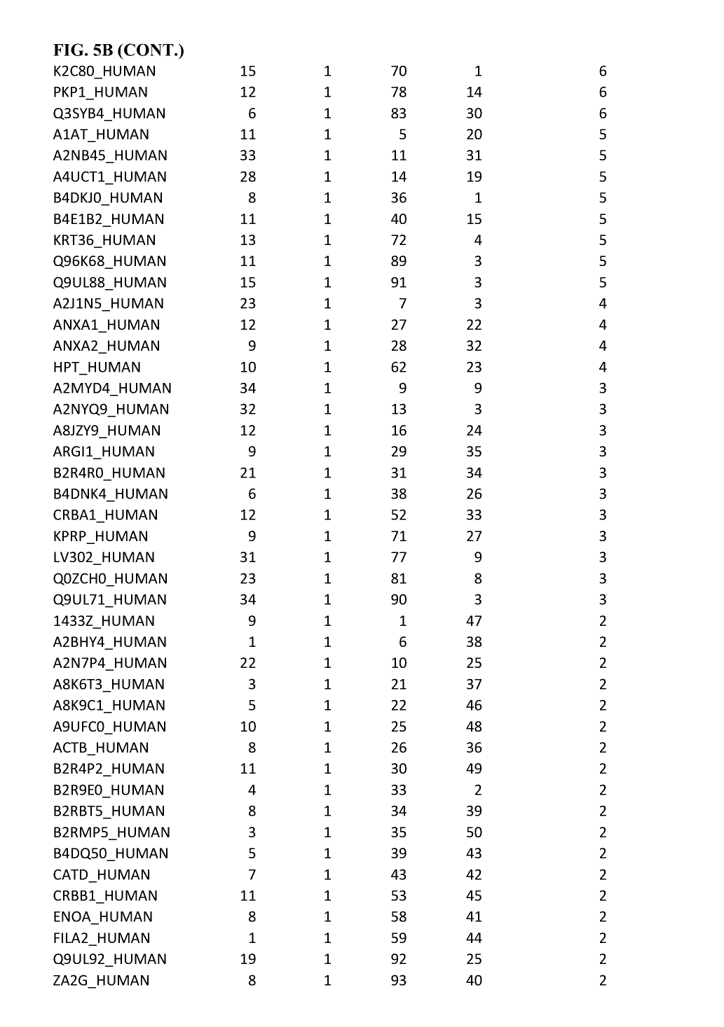

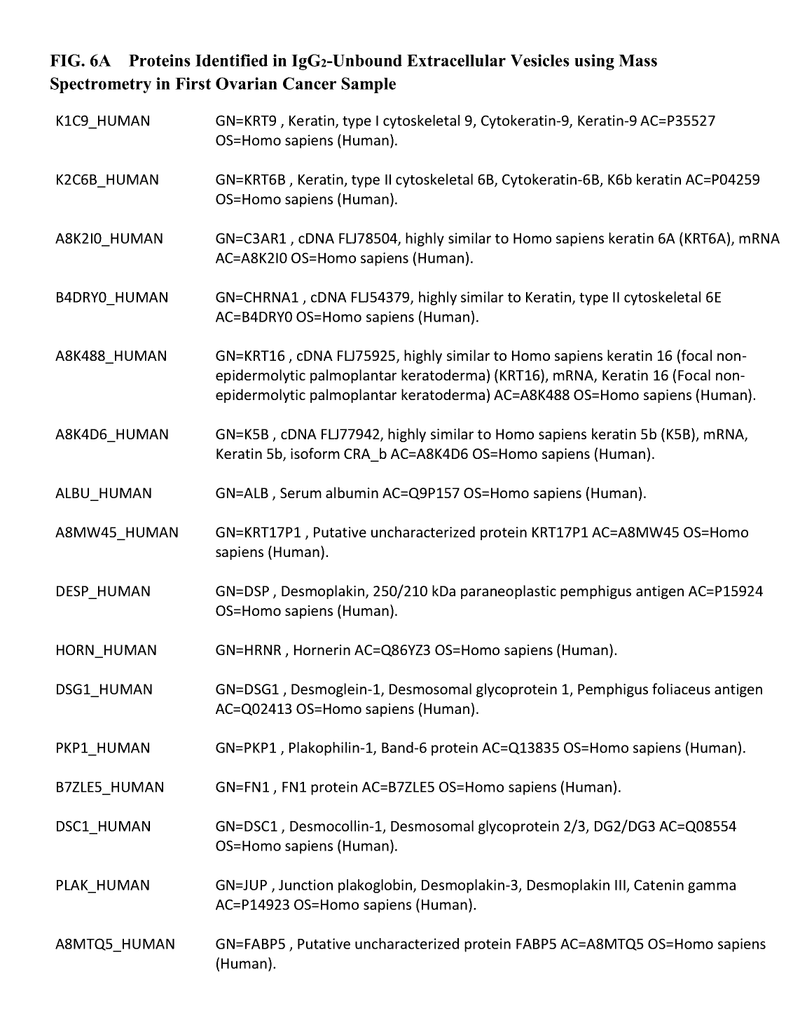

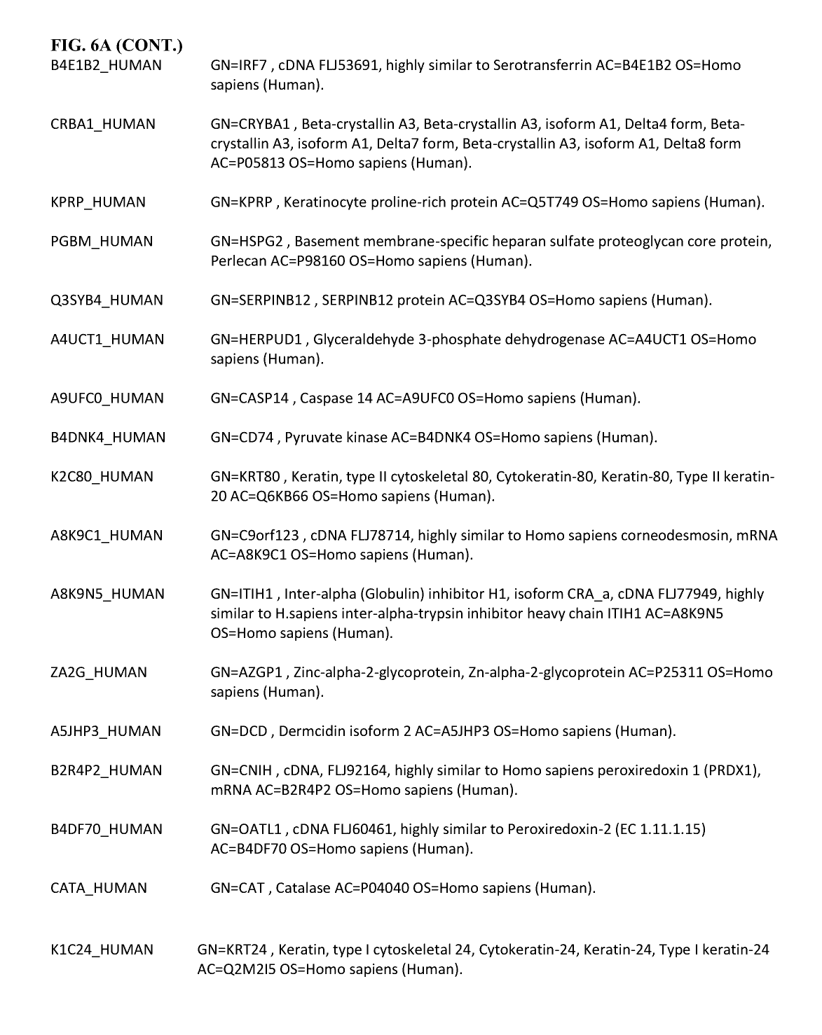

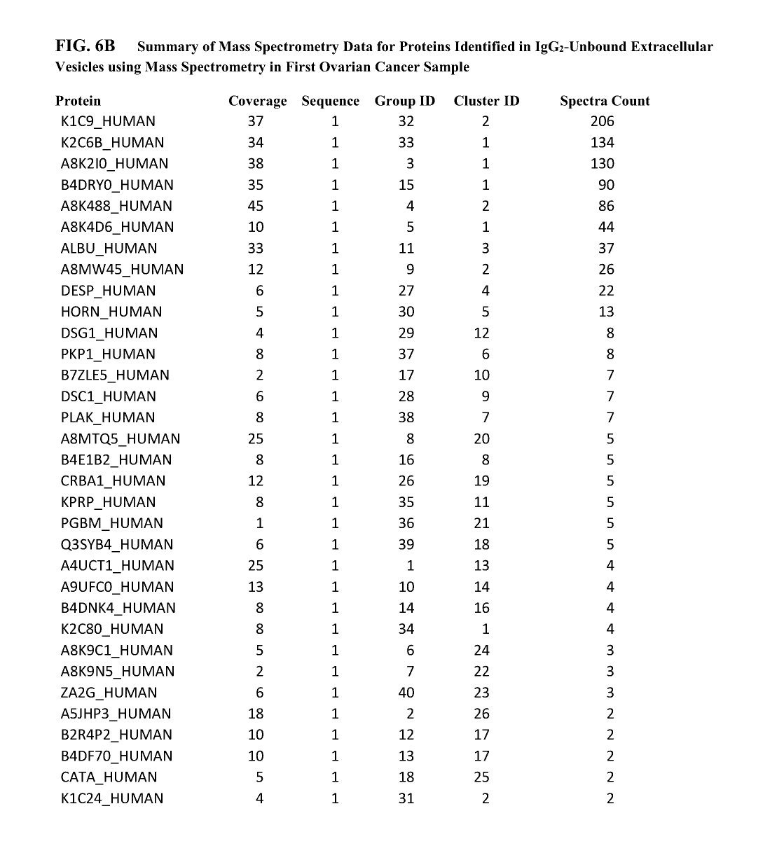

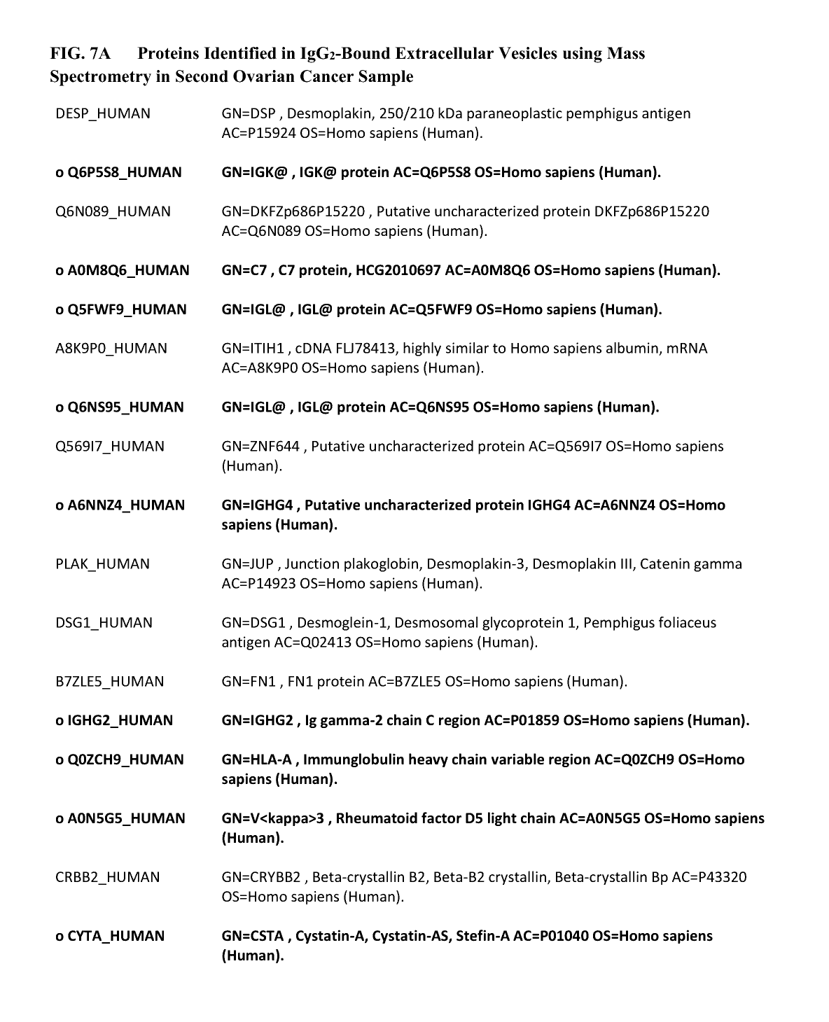

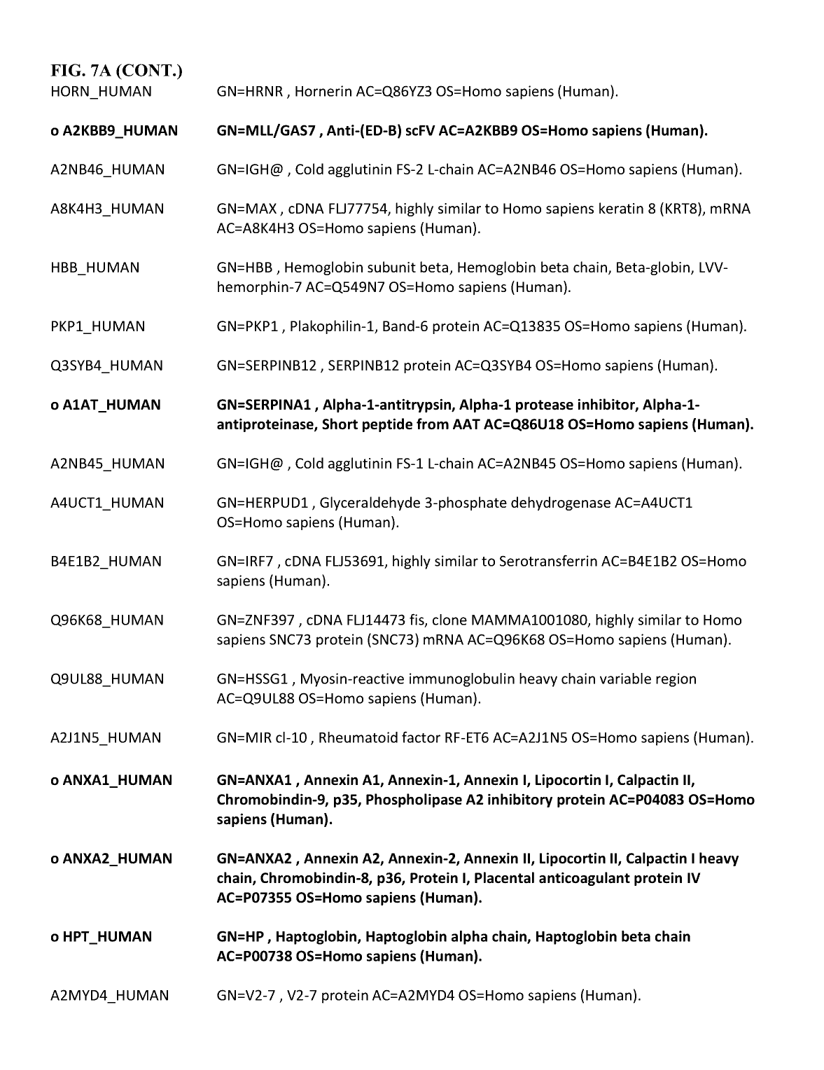

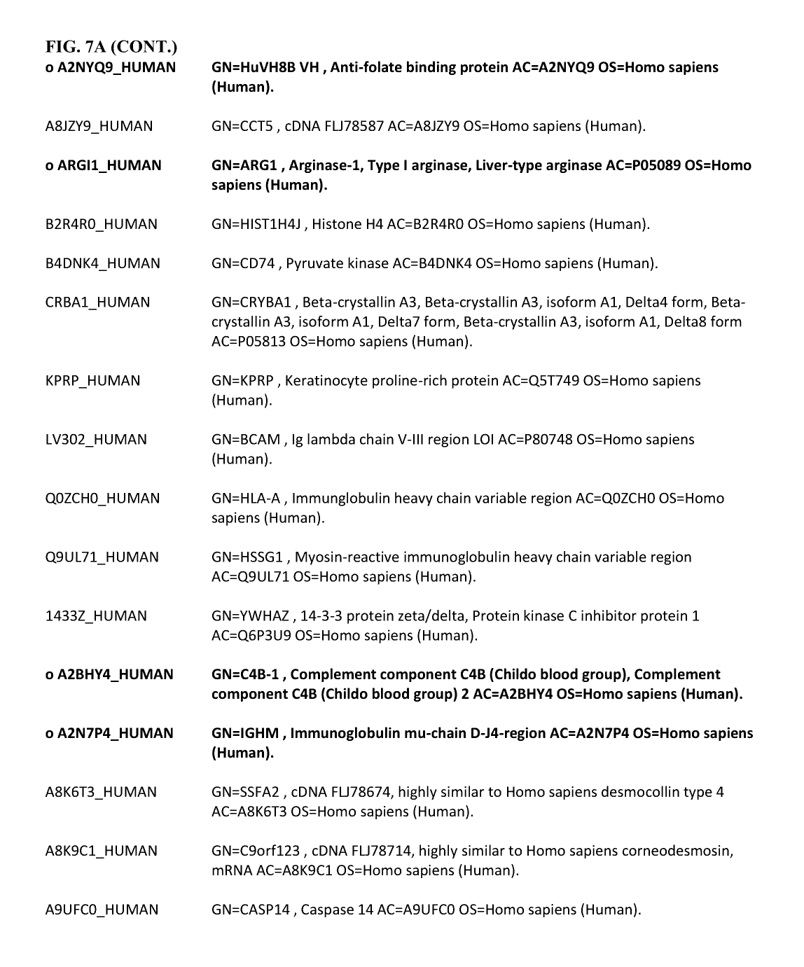

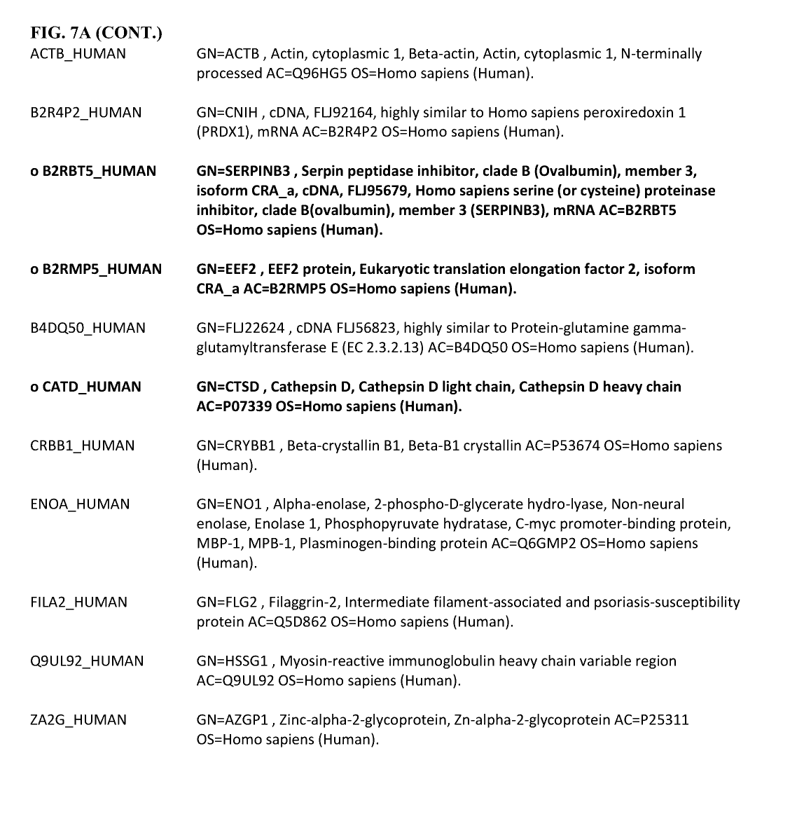

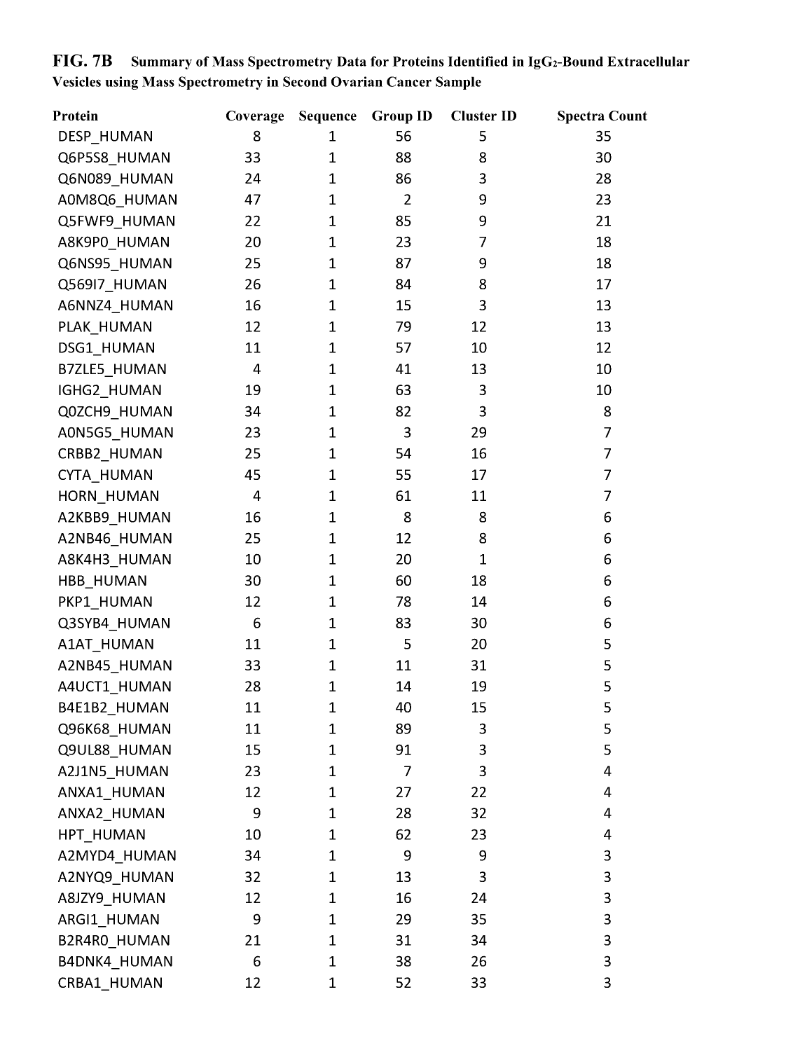

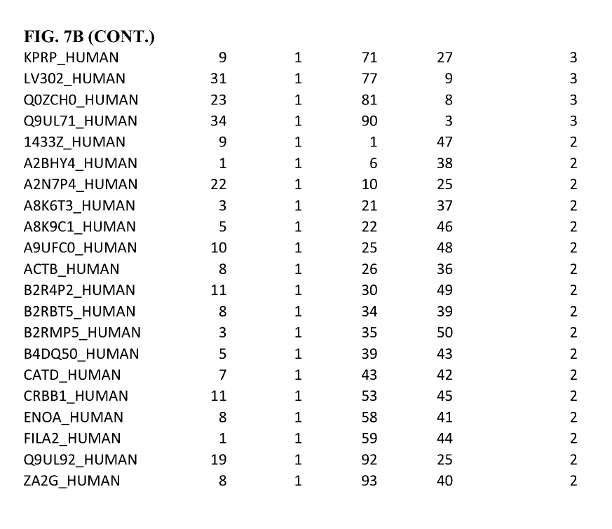

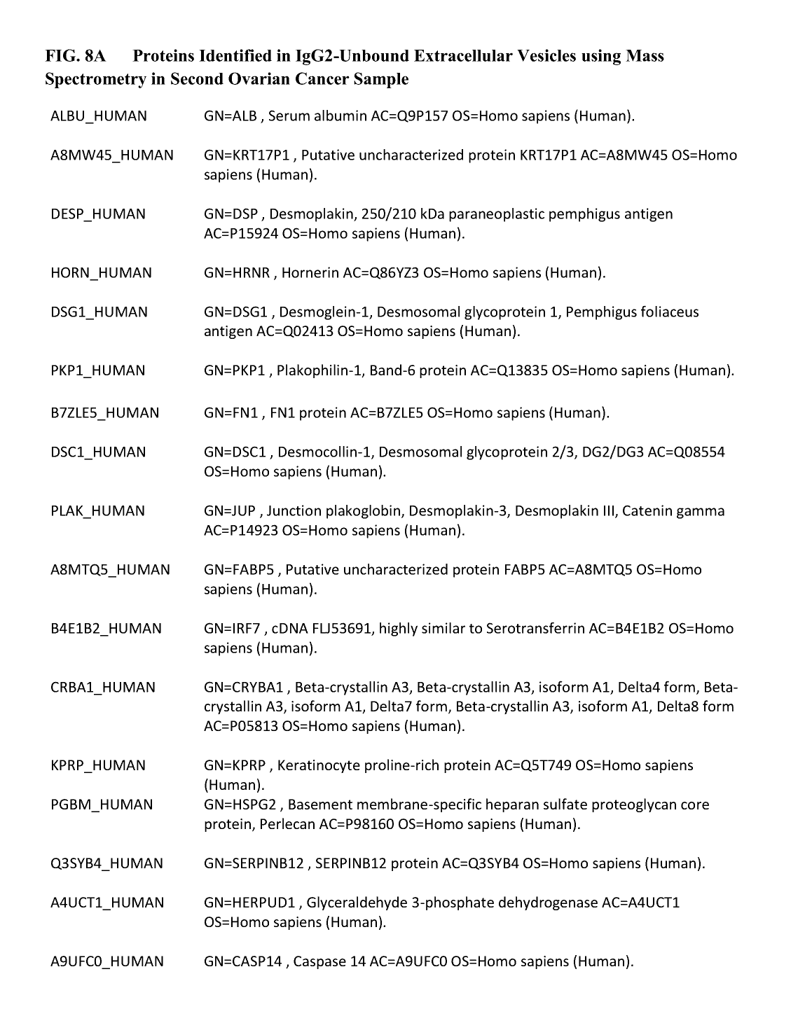

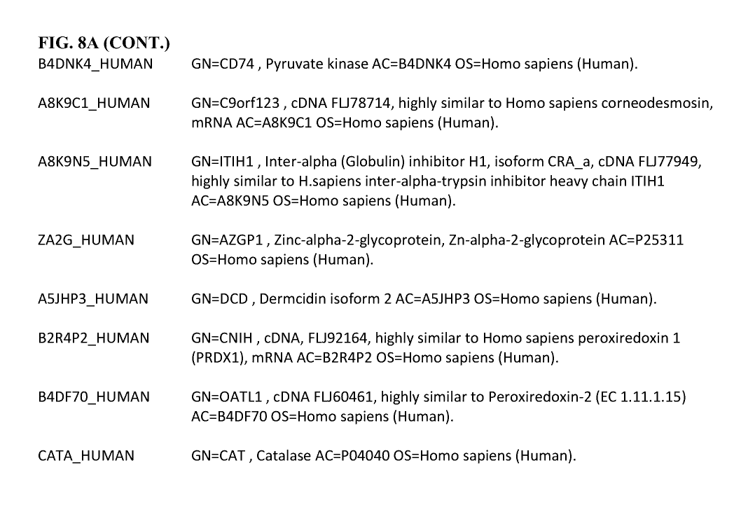

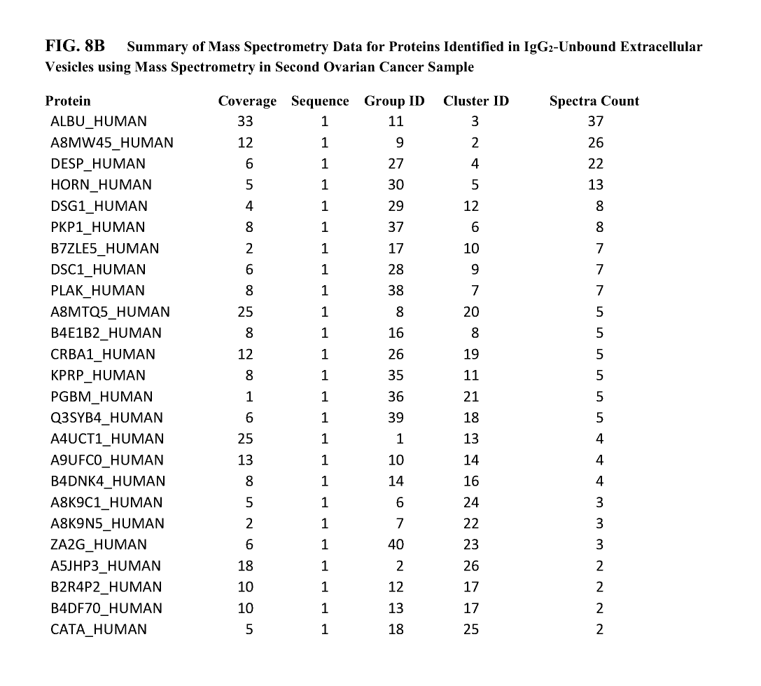

The list of proteins identified in the IgG-bound (e.g., IgG₂-bound or protein G-recognized IgG-bound) and the IgG-unbound (e.g., IgG₂-unbound or protein G-recognized IgG-unbound) extracellular vesicles from the First and Second Ovarian Cancer Subjects are listed in FIG. 5A, FIG. 6A, FIG. 7A, and FIG. 8A. A summary of the corresponding mass spectrometry data is shown in FIG. 5B, FIG. 6B, FIG. 7B, and FIG. 8B. The proteins uniquely detected in IgG-bound (e.g., IgG₂-bound or protein G-recognized IgG-bound) extracellular vesicles (i.e., not detected in IgG-unbound (e.g., IgG₂-unbound or protein G-recognized IgG-unbound) extacellular vesicles) are shown in bold in FIG. 5A and FIG. 7A. The proteins that were unique to the IgG-bound (e.g., IgG₂-bound or protein G-recognized IgG-bound) extracellular vesicles include C7, cystatin-A, alpha-1-antitrypsin, MIR-10, ANXA1, ANXA2, ARG-1, complement component C4B-1, haptoglobin, SERPINB3, EEF2, and CTSD. In addition, several IgG₂ proteins or fragments were detected in the IgG-bound (e.g., IgG₂-bound or protein G-recognized IgG-bound) extracellular vesicle population. The presence of IgG (e.g., IgG₂ or protein G-reactive IgG) proteins and fragments in the IgG-bound (e.g., IgG₂-bound or protein G-recognized IgG-bound) extracellular vesicle sample confirms that this sample contains IgG (e.g., IgG₂ or protein G-reactive IgG).

In sum, these data show that IgG-bound (e.g., IgG₂-bound or protein G-recognized IgG-bound) extracellular vesicles containing a variety of different tumor antigens can be isolated from a subject having cancer, and that subjects can be diagnosed as having a cancer based on the detection of one or more tumor antigens present in an isolated IgG (e.g., IgG₂-bound or protein G-recognized IgG-bound) extracellular vesicle.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of isolating a protein G-recognized IgG-bound extracellular vesicle, wherein the protein G-recognized IgG is bound to an antigen present on the surface of the extracellular vesicle and the protein G-recognized IgG is an endogenous antibody, the method comprising:
(a) obtaining a sample comprising a biological fluid from the subject,
(b) contacting the sample with an agent that specifically binds to a heavy chain constant domain of one or more protein G-recognized IgG,
(c) separating the agent bound with protein G-recognized IgG-bound extracellular vesicles from unbound material present in the sample, and
(d) dissociating the protein G-recognized IgG-bound extracellular vesicles from the agent, thereby isolating protein G-recognized IgG-bound extracellular vesicles.

2. The method of claim 1, wherein the protein G-recognized IgG is IgG₂.

3. The method of claim 1, wherein the agent is protein G.

4. The method of claim 1, wherein the agent is an antibody that specifically binds to a heavy chain constant domain of IgG₂.

5. The method of claim 1, wherein the biological fluid is blood, serum, plasma, urine, cerebrospinal fluid, saliva, tears, nasal discharge, semen, amniotic fluid, vaginal discharge, lymph, tears, mucus, synovial fluid, breast milk, vitreous humor, aqueous humor, or sweat.

6. The method of claim 1, further comprising, before the contacting in (b), enriching the sample for extracellular vesicles by passing the sample through a molecular sieve column or a molecular weight filter.

7. The method of claim 1, further comprising (e) centrifuging the eluate to generate a pellet, and (f) resuspending the pellet in a physiologically acceptable buffer.

8. The method of claim 1, wherein the sample is obtained from a subject having or suspected of having a cancer.

9. The method of claim 8, wherein the cancer is bladder cancer, epithelial cancer, prostate cancer, anal cancer, appendix cancer, bone cancer, brain tumor, breast cancer, heart cancer, cervical cancer, colon cancer, gallbladder cancer, stomach cancer, head and neck cancer, liver cancer, kidney cancer, laryngeal cancer, lung cancer, ovarian cancer, pancreatic cancer, penile cancer, pituitary cancer, rectal cancer, salivary gland cancer, sarcoma, testicular cancer, throat cancer, thyroid cancer, urethral cancer, uterine cancer, vaginal cancer, or vulvar cancer.

10. The method of claim 1, wherein the subject is human.

11. The method of claim 1, wherein the protein G-recognized IgG-bound extracellular vesicle contains at least one tumor antigen.

12. The method of claim 11, wherein the at least one tumor antigen is selected from the group consisting of: epidermal growth factor receptor (EGFR), complement 7, cystatin-A, alpha-1-antitrypsin, monocyte/macrophage Ig-related receptor-10, annexin A1, annexin A2, arginase-1, complement component C4B-1, haptoglobin, serpin peptidase inhibitor, clade B (Ovalbumin), member 3, eukaryotic translation elongation factor 2, and cathepsin D.

13. A method of diagnosing a cancer in a subject, the method comprising:
(a) obtaining a sample comprising a biological fluid from the subject;
(b) isolating a protein G-recognized IgG-bound extracellular vesicle from the sample, wherein the protein G-recognized IgG-bound extracellular vesicle contains a protein G-recognized IgG bound to an antigen present on the surface of the extracellular vesicle and the protein G-recognized IgG bound to the extracellular vesicle is an endogenous antibody; and
(c) detecting the presence of one or more tumor antigens in the isolated protein G-recognized IgG-bound extracellular vesicle; and
(d) diagnosing a subject having an isolated protein G-regonized IgG-bound extracellular vesicle containing one or more tumor antigens as having a cancer.

14. The method of claim 13, wherein the protein G-recognized IgG is IgG₂.

15. The method of claim 13, wherein the biological fluid is blood, serum, plasma, urine, cerebrospinal fluid, saliva, tears, nasal discharge, semen, amniotic fluid, vaginal discharge, lymph, tears, mucus, synovial fluid, breast milk, vitreous humor, aqueous humor, or sweat.

16. The method of claim 13, wherein in (d) a subject having a protein G-recognized-IgG-bound extracellular vesicle containing one of more tumor antigens selected from the group consisting of: epidermal growth factor receptor (EGFR), complement 7, cystatin-A, alpha-1-antitrypsin, monocyte/macrophage Ig-related receptor-10, annexin A1, annexin A2, arginase-1, complement component C4B-1, haptoglobin, serpin peptidase inhibitor, clade B (Ovalbumin), member 3, eukaryotic translation elongation factor 2, and cathepsin D is diagnosed with ovarian cancer.

17. The method of claim 13, wherein the isolating in (b) comprises contacting the sample with an agent that specifically binds to a heavy chain constant domain of IgG₂.

18. The method of claim 17, wherein the agent is protein G.

19. The method of claim 17, wherein the agent is an antibody that specifically binds to the heavy chain constant domain of IgG₂.

20. The method of claim 13, wherein the detecting in (c) is performed using mass spectrometry.

21. The method of claim 13, wherein the detecting in (c) is performed using two-dimensional gel electrophoresis.

22. The method of claim 13, wherein the detecting in (c) is performed using an exogenous antibody that specifically binds to a tumor antigen.

23. The method of claim 13, wherein the subject is a human.

24. The method of claim 13, further comprising administering to the subject diagnosed with cancer one or more cancer therapeutics.

25. A method of treating a subject, the method comprising selectively administering one or more cancer therapeutics to a subject that has been determined to have a protein G-recognized IgG-bound extracellular vesicle that contains one or more tumor antigens, wherein the protein G-recognized IgG-bound extracellular vesicle contains a protein G-recognized IgG bound to an antigen present on the surface of the extracellular vesicle and the protein G-recognized IgG bound to the extracellular vesicle is an endogenous antibody.

26. The method of claim 25, wherein the protein G-recognized IgG is IgG₂.

27. The method of claim 25, wherein the subject has been determined to have a protein G-recognized IgG-bound extracellular vesicle that contains one or more tumor antigens selected from the group consisting of: epidermal growth factor receptor (EGFR), complement 7, cystatin-A, alpha-1-antitrypsin, monocyte/macrophage Ig-related receptor-10, annexin A1, annexin A2, arginase-1, complement component C4B-1, haptoglobin, serpin peptidase inhibitor, clade B (Ovalbumin), member 3, eukaryotic translation elongation factor 2, and cathepsin D.

28. The method of claim 25, wherein the one or more cancer therapeutic is an antimetabolite, an alkylating agent, interleukin-2, or a therapeutic antibody.

29. The method of claim 25, wherein the administering is performed by oral, intravenous, intraarterial, subcutaneous, intramuscular, intraperitoneal, or intrathecal administration.

30. A method of selecting a subject for participation in a clinical trial, the method comprising:
(a) obtaining a sample comprising a biological fluid from a subject;
(b) isolating a protein G-recognized IgG-bound extracellular vesicle from the sample, wherein the protein G-recognized IgG-bound extracellular vesicle contains a protein G-recognized IgG bound to an antigen present on the surface of the extracellular vesicle and the protein G-recognized IgG bound to the extracellular vesicle is an endogenous antibody;
(c) detecting the presence of one or more tumor antigens in the isolated protein G-recognized IgG-bound extracellular vesicle; and
(d) selecting a subject having isolated protein G-recognized IgG-bound extracellular vesicles containing one or more tumor antigens for participation in a clinical study.

31. The method of claim 30, wherein the protein G-recognized IgG is IgG₂.

32. The method of claim 30, further comprising administering one or more cancer therapeutics to the subject during the clinical study.

33. The method of claim 30, wherein the biological fluid is blood, serum, plasma, urine, cerebrospinal fluid, saliva, tears, nasal discharge, semen, amniotic fluid, vaginal discharge, lymph, tears, mucus, synovial fluid, breast milk, vitreous humor, aqueous humor, or sweat.

34. The method of claim 30, wherein the isolating in (b) comprises contacting the sample with an agent that specifically binds to a heavy chain constant domain of IgG₂.

35. The method of claim 34, wherein the agent is protein G.

36. The method of claim 34, wherein the agent is an antibody that specifically binds to the heavy chain constant domain of IgG₂.

37. The method of claim 30, wherein the detecting in (c) is performed using mass spectrometry.

38. The method of claim 30, wherein the detecting in (c) is performed using two-dimensional gel electrophoresis.

39. The method of claim 30, wherein the detecting in (c) is performed using an exogenous antibody that specifically binds to a tumor antigen.

40. The method of claim 30, wherein the subject is a human.
